(19) United States
(12) Patent Application Publication
(10) Pub. No.: US 2019/0328691 A1
(43) Pub. Date: Oct. 31, 2019

(54) ANTIBIOTIC THERAPY

(71) Applicant: Boulos & Cooper Pharmaceuticals Pty Ltd, Balcatta (AU)

(72) Inventor: Ramiz Boulos, Banksia Grove (AU)

(21) Appl. No.: 15/760,012

(22) PCT Filed: Aug. 29, 2016
(86) PCT No.: PCT/AU2016/050807

§ 371 Date: Mar. 14, 2018

(30) Foreign Application Priority Data
Sep. 14, 2015 (AU) 2015903731

(51) Int. Cl. A61K 31/194; A61K 31/546; A61K 31/431; A61K 38/14; A61K 31/5377; A61K 38/12; A61K 31/351; A61P 31/04

(52) U.S. Cl. CPC A61K 31/194; A61K 31/546; A61K 31/431; A61P 31/04; A61K 31/5377; A61K 38/12; A61K 31/351; A61K 38/14

(57) ABSTRACT

The present invention provides a composition comprising a combination of: (i) at least one aryl antibiotic comprising a compound having a structure selected from Group I, or a pharmaceutically acceptable salt thereof; and (ii) at least one antibiotic selected from the list comprising a penicillin or a derivative thereof, a cepham or a derivative thereof, vancomycin, linezolid, daptomycin and mupirocin.

### TECHNICAL FIELD

An antibacterial composition comprising a combination of an aryl antibiotic or a pharmaceutically acceptable salt thereof and an antibiotic chosen from the list comprising a penicillin or a derivative thereof, a cepham or a derivative thereof, vancomycin, linezolid, daptomycin and mupirocin, useful for the treatment or prevention of bacterial infection, the control or manipulation of commensal bacterial populations and for the prevention, control or removal of bacterial contamination.

### BACKGROUND ART

Combination antibiotic therapy can have a number of advantages over traditional monotherapy including but not limited to, higher rate of treatment success, and slower development of resistance. The slower emergence of resistance is a direct result of the lower likelihood of pathogens developing resistance to two different drugs, especially when they have different mechanism of action and independent targets.

In some cases, a pathogen may have developed multi-drug resistance to a number of antibiotics and combination therapy then becomes a preferred treatment option to the inactivity of each antibiotic alone. In such cases, an additive or sub-additive effect is sufficient for initiating antibiotic combination therapy, as treatment options become limited.

However, it is difficult to predict which antibiotics may be used in combination. For example, Johansen et al (2000) [J. Antimicrob. Chemother. 46:973-980) found that a combination of penicillin and erythromycin caused increased mortality due to antagonism between the antibiotics; and Thauvin et al (1985) [Antimicrob. Agents Chemother. 28:78-83] found that a penicillin amikacin combination was antagonistic in activity.

Recently, strains of Staphylococcus aureus have acquired resistance to daptomycin and linezolid, two of newest FDA approved antibiotics that were previously able to fight serious infections caused by S. aureus successfully.

There is no indication that bacterial resistance to antibiotics will stop and for this reason new antibiotics and new treatment options are necessary to achieve a desirable treatment outcome in patients.

The above discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a composition comprising a combination of: (i) at least one aryl antibiotic comprising a compound having a structure selected from Group I, wherein Group I consists of:

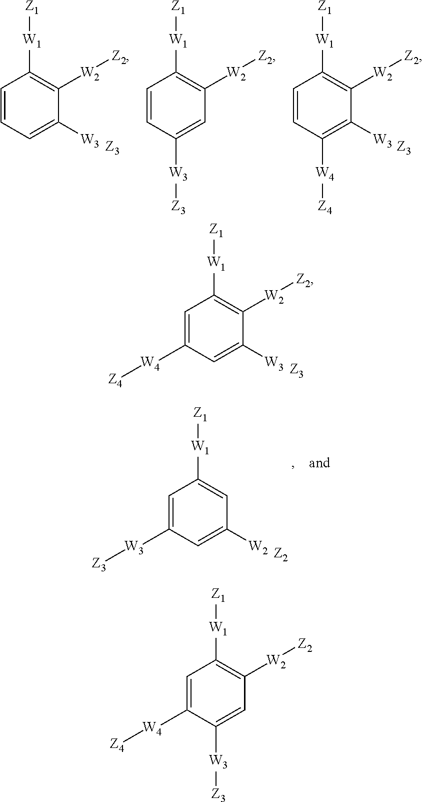

wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of:

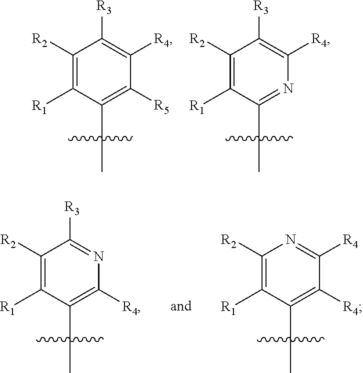

each of R₁, R₂, R₃, R₄, and R₅ is independently C₁₋₈ heteroalkyl, and the C₁₋₈ heteroalkyl comprises CO₂H or an ester thereof, with the proviso that at least one of R₁, R₂, R₃, R₄, and R₅ is C₁₋₈ heteroalkyl, or a pharmaceutically acceptable salt thereof; and (ii) at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

The present invention provides a composition comprising a combination of: (i) at least one aryl antibiotic of any one of:

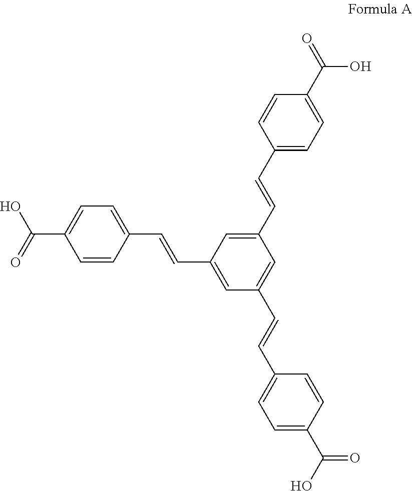 [SMILES: O=C(O)c1ccc(/C=C/c2cc(/C=C/c3ccc(C(=O)O)cc3)cc(/C=C/c3ccc(C(=O)O)cc3)c2)cc1]

or pharmaceutically acceptable salts thereof; and/or Formula B:

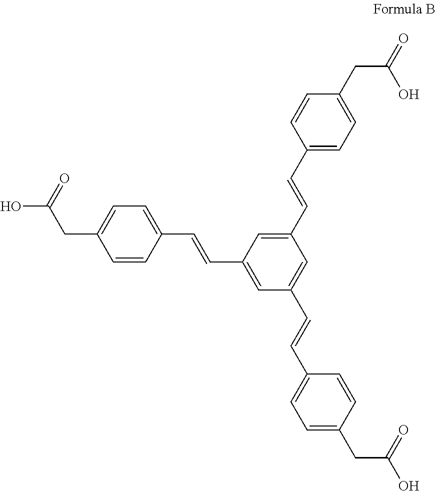 [SMILES: O=C(O)Cc1ccc(/C=C/c2cc(/C=C/c3ccc(CC(=O)O)cc3)cc(/C=C/c3ccc(CC(=O)O)cc3)c2)cc1]

or pharmaceutically acceptable salts thereof; and/or Formula C:

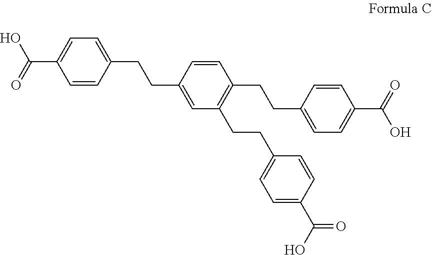 [SMILES: O=C(O)c1ccc(CCc2ccc(CCc3ccc(C(=O)O)cc3)c(CCc3ccc(C(=O)O)cc3)c2)cc1]

or pharmaceutically acceptable salts thereof; and/or Formula D:

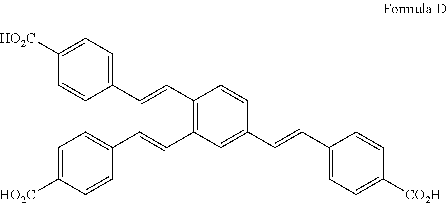 [SMILES: O=C(O)c1ccc(/C=C/c2ccc(/C=C/c3ccc(C(=O)O)cc3)c(/C=C/c3ccc(C(=O)O)cc3)c2)cc1]

or pharmaceutically acceptable salts thereof; and/or Formula E:

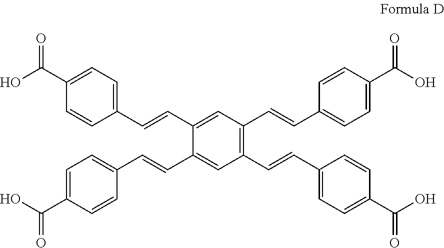 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

or pharmaceutically acceptable salts thereof; and (ii) at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

Preferably, the combination of an aryl antibiotic with at least one antibiotic selected from the list comprising a penicillin or a derivative thereof, a cepham or a derivative thereof, vancomycin, linezolid, daptomycin and mupirocin has additive or synergistic activity; more preferably synergistic activity.

The present invention further provides a method for suppressing, inhibiting, preventing, alleviating or treating a bacterial infection, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.

Preferably the antibiotic of (ii) is a penicillin or a derivative thereof or a cepham or a derivative thereof.

The present invention further provides a method to control or manipulate a commensal bacterial population, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.

Preferably the antibiotic of (ii) is a penicillin or a derivative thereof or a cepham or a derivative thereof.

The present invention further provides a method to delay or prevent the development of antibiotic resistance in a bacteria to the aryl antibiotics of Group I or pharmaceutically acceptable salts thereof and/or the antibiotics selected from the list comprising a penicillin or a derivative thereof, a cepham or a derivative thereof, vancomycin, linezolid, daptomycin and mupirocin, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.

Preferably the antibiotic of (ii) is a penicillin or a derivative thereof or a cepham or a derivative thereof.

The present invention further provides a method to prevent, remove or control bacterial contamination, the method comprising the step of:
a) applying a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.

Preferably the antibiotic of (ii) is a penicillin or a derivative thereof or a cepham or a derivative thereof.

The present invention further provides for the use of at least one aryl antibiotic of Group I or pharmaceutically acceptable salts thereof for the manufacture of a medicament for the treatment of a bacterial infection, in combination with at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

Preferably, the bacteria is a Staphylococcus species.

The present invention further provides a pharmaceutical composition comprising a combination of: (i) at least one aryl antibiotic of Group I or pharmaceutically acceptable salts thereof; and (ii) at least one antibiotic selected from the list comprising a penicillin or a derivative thereof, a cepham or a derivative thereof, vancomycin, linezolid, daptomycin and mupirocin and one or more excipients. Preferably the antibiotic of (ii) is a penicillin or a derivative thereof or a cepham or a derivative thereof.

Preferably, the penicillin or a derivative thereof is oxacillin.

Preferably, cepham or a derivative thereof is cefepime.

### DESCRIPTION OF THE INVENTION

### Detailed Description of the Invention

The present invention provides a composition comprising a combination of antibiotics that act together to control bacterial growth, for example to treat bacterial infections or remove bacterial contamination.

The present invention provides a composition comprising a combination of: (i) at least one aryl antibiotic comprising a compound having a structure selected from Group I, wherein Group I consists of:

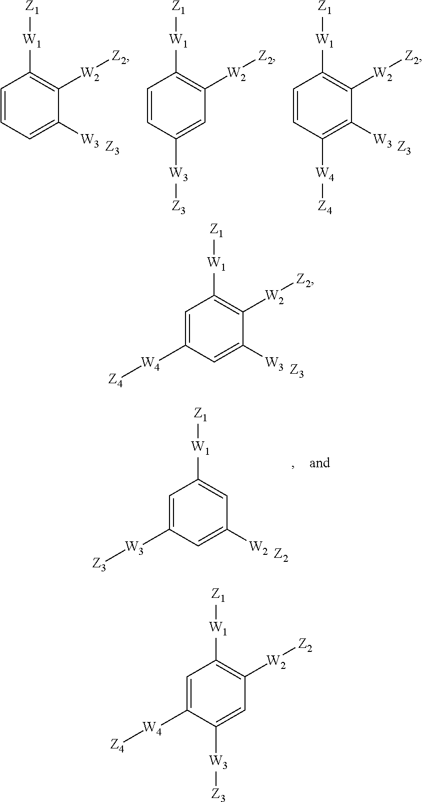

wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of:

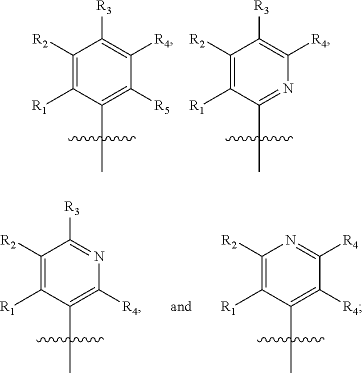

each of R₁, R₂, R₃, R₄, and R₅ is independently C₁₋₈ heteroalkyl, and the C₁₋₈ heteroalkyl comprises CO₂H or an ester thereof, with the proviso that at least one of R₁, R₂, R₃, R₄, and R₅ is C₁₋₈ heteroalkyl,or a pharmaceutically acceptable salt thereof; and (ii) at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

Preferably, in the compounds of Group I, each of W₁, W₂, W₃, and W₄ is a C₂ alkene.

Preferably, in the compounds of Group I, the C₂ alkene is in an E configuration.

Preferably, in the compounds of Group I, the C₂ alkene is in a Z configuration.

Preferably, the compound of Group I is Formula A:

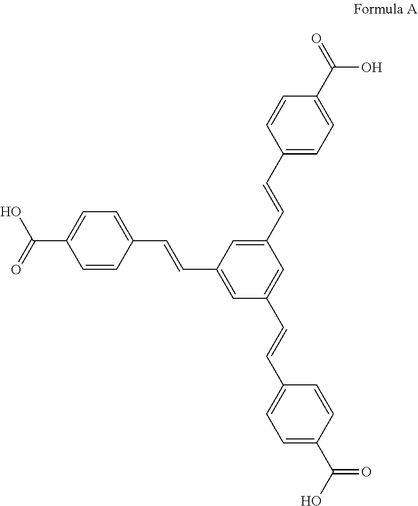 [SMILES: O=C(O)c1ccc(/C=C/c2cc(/C=C/c3ccc(C(=O)O)cc3)cc(/C=C/c3ccc(C(=O)O)cc3)c2)cc1]

or a pharmaceutically acceptable salt thereof.

Preferably, the compound of Group I is Formula B:

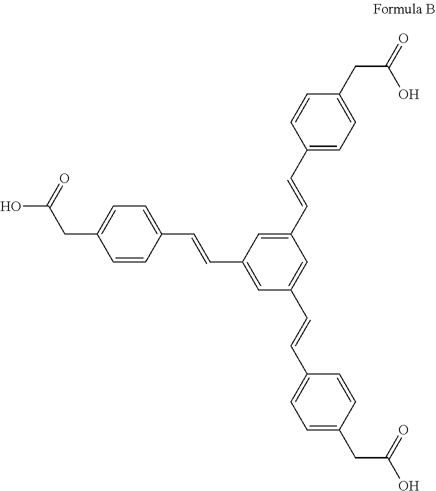 [SMILES: O=C(O)Cc1ccc(/C=C/c2cc(/C=C/c3ccc(CC(=O)O)cc3)cc(/C=C/c3ccc(CC(=O)O)cc3)c2)cc1]

or pharmaceutically acceptable salts thereof.

Preferably, the compound of Group I is Formula C:

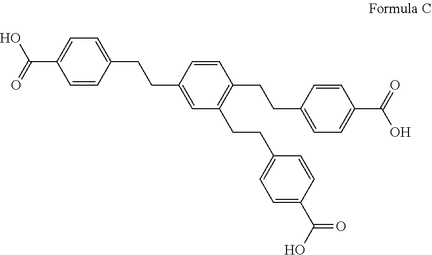 [SMILES: O=C(O)c1ccc(CCc2ccc(CCc3ccc(C(=O)O)cc3)c(CCc3ccc(C(=O)O)cc3)c2)cc1]

or pharmaceutically acceptable salts thereof.

Preferably, the compound of Group I is Formula D:

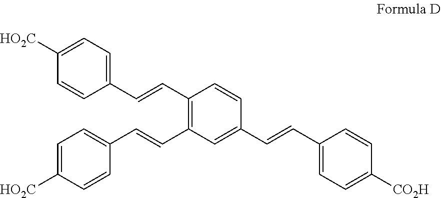 [SMILES: O=C(O)c1ccc(/C=C/c2ccc(/C=C/c3ccc(C(=O)O)cc3)c(/C=C/c3ccc(C(=O)O)cc3)c2)cc1]

or pharmaceutically acceptable salts thereof.

Preferably, the compound of Group I is Formula E:

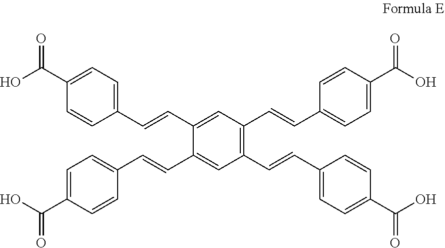 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

or pharmaceutically acceptable salts thereof.

The present invention provides a composition comprising a combination of: (i) at least one aryl antibiotic of any one of:

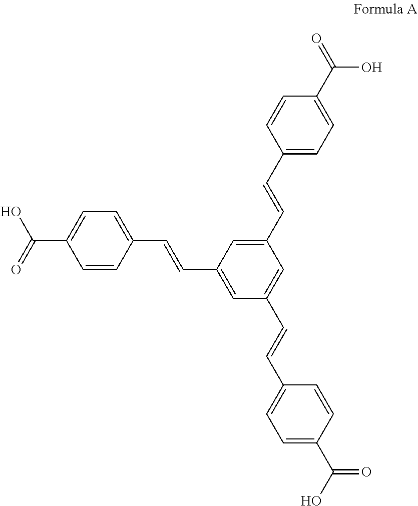 [SMILES: O=C(O)c1ccc(/C=C/c2cc(/C=C/c3ccc(C(=O)O)cc3)cc(/C=C/c3ccc(C(=O)O)cc3)c2)cc1]

or pharmaceutically acceptable salts thereof; and/or Formula B:

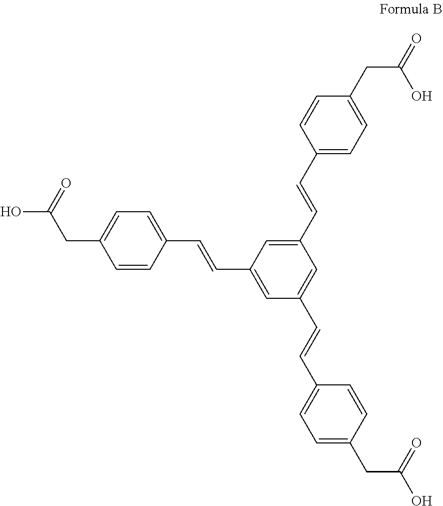 [SMILES: O=C(O)Cc1ccc(/C=C/c2cc(/C=C/c3ccc(CC(=O)O)cc3)cc(/C=C/c3ccc(CC(=O)O)cc3)c2)cc1]

or pharmaceutically acceptable salts thereof; and/or Formula C:

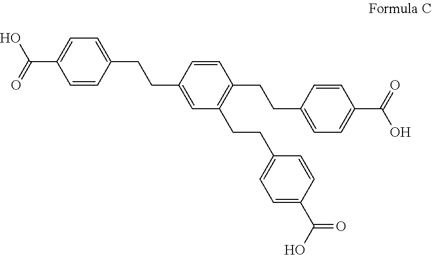 [SMILES: O=C(O)c1ccc(CCc2ccc(CCc3ccc(C(=O)O)cc3)c(CCc3ccc(C(=O)O)cc3)c2)cc1]

or pharmaceutically acceptable salts thereof; and/or Formula D:

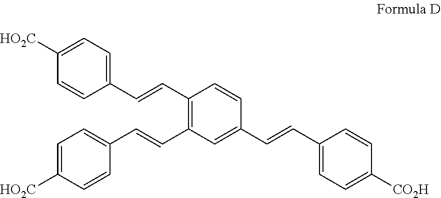 [SMILES: O=C(O)c1ccc(/C=C/c2ccc(/C=C/c3ccc(C(=O)O)cc3)c(/C=C/c3ccc(C(=O)O)cc3)c2)cc1]

or pharmaceutically acceptable salts thereof; and/or Formula E:

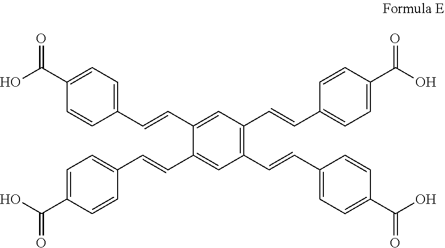 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

or pharmaceutically acceptable salts thereof; and (ii) at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

Preferably, the present invention provides a composition comprising a combination of: (i) at least one aryl antibiotic of Formula A or Formula B, or pharmaceutically acceptable salts thereof; and (ii) at least one antibiotic selected from the list comprising: a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

Preferably, the combination of an aryl antibiotic with at least one antibiotic selected from the list comprising a penicillin or a derivative thereof, a cepham or a derivative thereof, vancomycin, linezolid, daptomycin and mupirocin has additive or synergistic activity to suppress, inhibit, prevent, alleviate or treat a bacterial infection, control or manipulate a bacterial population, delay or prevent the development of antibiotic resistance in bacteria and/or prevent, control or remove bacterial contamination than the aryl antibiotic alone or the penicillin or penicillin derivative, cepham or cepham derivative, vancomycin, linezolid, daptomycin or mupirocin alone, when used at the same dosage.

The composition according to the present invention may be characterized by a synergistic effect. Without wishing to be bound by theory, the additive or synergistically enhanced of activity by the aryl antibiotic may be explained by its mode of action, involving the opening of the MscL channel of the microorganisms.

Such compositions are useful for suppressing, inhibiting, preventing, alleviating or treating a bacterial infection, more particularly a bacterial infection caused by Gram-positive bacteria. The terms “suppressing”, “inhibiting”, “preventing”, “alleviating” or “treating” are defined herein to mean delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics.

The compositions may further or additionally be used in the control or manipulation of commensal bacterial populations. For example, the compositions may be used to control or manipulate a bacterial population in subject that is about to undergo surgery, or become immunocompromised through, for example, chemotherapy. Such subjects may not have an active infection, but may carry a normal microbial population that includes bacteria that may become problematic and cause infections after the surgery or immunocompromising event. It may be advantageous to eliminate or at least reduce the numbers of one or more bacterial species that form the normal body flora of the subject before the surgery or immunocompromising event, such that the bacteria is unable to take advantage of the event and cause an infection. A commensal bacterial population is the normal microflora or indigenous microbiota of a subject, consisting of those micro-organisms which are normally present on body surfaces covered by epithelial cells and are exposed to the external environment (gastrointestinal and respiratory tract, vagina, skin, etc). By eliminate or reduce, it is meant that the number of one or more chosen species for bacteria are reduced in numbers compared to before administration of the compositions of the invention, preferably to the level where they are either no longer present, or at least are present in very low numbers, preferably lower than the number of bacteria needed to establish an infection.

The development of antibiotic resistance is an increasing issue in the fields of therapeutic treatment of subjects and environmental contamination control. One of the ways to address the development of resistance is to administer two or more antibiotics in conjunction, in order to reduce the chance that bacteria will be able to develop resistance to either active agent.

The compositions may further or additionally be used in methods to prevent bacterial growth, stop bacterial growth and/or killing bacterial cells and prevent, control or remove bacterial contamination. Methods using the compositions of the invention can be performed in vivo, ex vivo, in vitro, etc. For example, the compositions of the invention may be used to sterilize devices and/or compositions (such as medical and/or dental equipment, devices, and/or compositions) to prevent, control or remove bacterial contamination. Methods using the compositions of the invention may also be useful for sterilizing facilities at medical and/or dental centres (e.g., hospital rooms, operating rooms, emergency rooms, etc) to prevent, control or remove bacterial contamination.

The aryl antibiotics of Group I or pharmaceutically acceptable salts thereof; and/or the antibiotics selected from the list comprising penicillin or a derivative thereof, cepham or a derivative thereof, vancomycin, linezolid, daptomycin or mupirocin may be referred to herein as “the compounds”, “the antibiotics”, “the antibiotic compounds”, “the actives”, and/or “the active agents”. Reference to the compounds etc may refer to the antibiotics of Group I, the antibiotics of Formula A, antibiotics of Formula B, antibiotics of Formula C, antibiotics of Formula D, antibiotics of Formula E, the penicillin or penicillin derivatives, the cepham or cepham derivatives, vancomycin, linezolid, daptomycin or mupirocin either alone or any combination of two or more of these.

Preferably, the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin thereof work in an additive or synergistic manner to suppress, inhibit, prevent, alleviate or treat a bacterial infection. Preferably the antibiotic of (ii) is a penicillin or a derivative thereof or a cepham or a derivative thereof.

By additive, it is meant that the effect of two or more components together is at least the same as the sum of their effects when used individually. Synergism or a synergistic effect refers to a phenomenon whereby the effect of two or more components together is greater than the sum of their effects when used individually.

There are many ways to test the relative activity of two or more compounds in combination. For example, the FIC (Fraction Inhibitory Concentration) and FIC Index (FICI) values for each test condition and the average of FICI values of all pairwise combinations can be calculated to determine if synergy or antagonism existed between two substances. The FICI may be determined by calculating the sum of the ratios of Minimum Inhibitory Concentrations (MICs) for both substances. Arithmetically, the FIC Index of a combination of Substance 1 and Substance 2 may be defined as following: FICI=Σ[FIC(Substance 1)+FIC(Substance 2)]=[(MIC of Substance 1 in combination/MIC of Substance 1 alone)+(MIC of Substance 2 in combination/MIC of Substance 2 alone)]. By this definition, Synergy is defined as the FICI (Σ)≤0.5; additivity as FICI (Σ)>0.5 to ≤1; indifference as FICI (Σ)>1 to ≤4; and antagonism is defined as the FICI (Σ)>4.

More preferably, the aryl antibiotics of Group I or pharmaceutically acceptable salts thereof and the antibiotic selected from the list comprising a penicillin or a derivative thereof, a cepham or a derivative thereof, vancomycin, linezolid, daptomycin and mupirocin work in a synergistic manner to suppress, inhibit, prevent, alleviate or treat a bacterial infection. Preferably the antibiotic of (ii) is a penicillin or a derivative thereof or a cepham or a derivative thereof.

The (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in an additive or synergistic manner to control or manipulate a bacterial population. Preferably the antibiotic of (ii) is a penicillin or a derivative thereof or a cepham or a derivative thereof.

More preferably, the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in a synergistic manner to control or manipulate a bacterial population. Preferably the antibiotic of (ii) is a penicillin or a derivative thereof or a cepham or a derivative thereof.

The (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in an additive or synergistic manner to delay or prevent the development of antibiotic resistance in a bacterial population. Preferably the antibiotic of (ii) is a penicillin or a derivative thereof or a cepham or a derivative thereof.

More preferably, the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in a synergistic manner to delay or prevent the development of antibiotic resistance in a bacterial population. Preferably the antibiotic of (ii) is a penicillin or a derivative thereof or a cepham or a derivative thereof.

The (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in an additive or synergistic manner to prevent, control or remove bacterial contamination. Preferably the antibiotic of (ii) is a penicillin or a derivative thereof or a cepham or a derivative thereof.

More preferably, the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin work in a synergistic manner to prevent, control or remove bacterial contamination. Preferably the antibiotic of (ii) is a penicillin or a derivative thereof or a cepham or a derivative thereof.

This additive or synergistic enhanced antibacterial activity allows the composition to have potent efficacy against a wide range of bacteria. Preferably, the antibacterial activity may be enhanced at levels where the antibiotics used individually may not be as effective.

Preferably, the present invention provides:
a composition comprising a combination of: (i) at least one aryl antibiotic of Group I or pharmaceutically acceptable salts thereof; and (ii) a penicillin or a derivative thereof;
a composition comprising a combination of: (i) at least one aryl antibiotic of Group I or pharmaceutically acceptable salts thereof; and (ii) a cepham or a derivative thereof;
a composition comprising a combination of: (i) at least one aryl antibiotic of Group I or pharmaceutically acceptable salts thereof; and (ii) vancomycin;
a composition comprising a combination of: (i) at least one aryl antibiotic of Group I or pharmaceutically acceptable salts thereof; and (ii) linezolid;
a composition comprising a combination of: (i) at least one aryl antibiotic of Group I or pharmaceutically acceptable salts thereof; and (ii) daptomycin; and/or
a composition comprising a combination of: (i) at least one aryl antibiotic of Group I or pharmaceutically acceptable salts thereof; and (ii) mupirocin.

Preferably, the present invention provides:
a composition comprising a combination of: (i) at least one aryl antibiotic of Formula A or Formula B or pharmaceutically acceptable salts thereof; and (ii) an antibiotic chosen from the list comprising a penicillin or a derivative thereof, a cepham or a derivative thereof, vancomycin, linezolid, daptomycin and mupirocin;
a composition comprising a combination of: (i) at least one aryl antibiotic of Formula A or Formula B or pharmaceutically acceptable salts thereof; and (ii) a penicillin or a derivative thereof;
a composition comprising a combination of: (i) at least one aryl antibiotic of Formula A or Formula B or pharmaceutically acceptable salts thereof; and (ii) a cepham or a derivative thereof;
a composition comprising a combination of: (i) at least one aryl antibiotic of Formula A or Formula B or pharmaceutically acceptable salts thereof; and (ii) vancomycin;
a composition comprising a combination of: (i) at least one aryl antibiotic of Formula A or Formula B or pharmaceutically acceptable salts thereof; and (ii) linezolid;
a composition comprising a combination of: (i) at least one aryl antibiotic of Formula A or Formula B or pharmaceutically acceptable salts thereof; and (ii) daptomycin; and/or
a composition comprising a combination of: (i) at least one aryl antibiotic of Formula A or Formula B or pharmaceutically acceptable salts thereof; and (ii) mupirocin;
a composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula A or pharmaceutically acceptable salts thereof; and (ii) at least one antibiotic selected from the list comprising vancomycin, linezolid, daptomycin and mupirocin;
a composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula B or pharmaceutically acceptable salts thereof; and (ii) at least one antibiotic selected from the list comprising vancomycin, linezolid, daptomycin and mupirocin;
a composition comprising a combination of: (i) an aryl antibiotic of Formula A or pharmaceutically acceptable salts thereof; and (ii) at least one penicillin or a derivative thereof;
a composition comprising a combination of: (i) an aryl antibiotic of Formula A or pharmaceutically acceptable salts thereof; and (ii) at least one cepham or a derivative thereof;
a composition comprising a combination of: (i) an aryl antibiotic of Formula A or pharmaceutically acceptable salts thereof; and (ii) vancomycin;
a composition comprising a combination of: (i) an aryl antibiotic of Formula A or pharmaceutically acceptable salts thereof; and (ii) linezolid;
a composition comprising a combination of: (i) an aryl antibiotic of Formula A or pharmaceutically acceptable salts thereof; and (ii) daptomycin;
a composition comprising a combination of: (i) an aryl antibiotic of Formula A or pharmaceutically acceptable salts thereof; and (ii) mupirocin;
a composition comprising a combination of: (i) an aryl antibiotic of Formula B or pharmaceutically acceptable salts thereof; and (ii) at least one penicillin or a derivative thereof;
a composition comprising a combination of: (i) an aryl antibiotic of Formula B or pharmaceutically acceptable salts thereof; and (ii) at least one cepham or a derivative thereof;
a composition comprising a combination of: (i) an aryl antibiotic of Formula B or pharmaceutically acceptable salts thereof; and (ii) vancomycin;
a composition comprising a combination of: (i) an aryl antibiotic of Formula B or pharmaceutically acceptable salts thereof; and (ii) linezolid;
a composition comprising a combination of: (i) an aryl antibiotic of Formula B or pharmaceutically acceptable salts thereof; and (ii) daptomycin; and/or
a composition comprising a combination of: (i) an aryl antibiotic of Formula B or pharmaceutically acceptable salts thereof; and (ii) mupirocin.

The mechanosensitive ion channel of large conductance (MscL) is a highly conserved transmembrane protein found in most bacterial species. It acts as an emergency valve in times of an osmotic downshock and, by doing so, prevents the cell from lysing. More importantly it is not present in the human genome, making it an ideal drug target. International patent application WO 2012/075766 describes a series of novel aryl compounds and their use as antibiotics to treat bacterial infections or diseases that act partly via the MscL. Without being bound by theory, it is believed that the aryl antibiotics work by lowering the threshold to open the channel and at the same time prolong the channel opening. The result is loss of osmolytes and solutes via the open channel which eventually lead to bacterial cell death. Furthermore more frequent opening of the MscL channel may facilitate the passage of other antibiotics into the bacterial cell.

The aryl antibiotic of Group I to be combined with the antibiotic chosen from the list comprising penicillin or a derivative thereof, a cepham or a derivative thereof, vancomycin, linezolid, daptomycin and mupirocin in the compositions of the present invention are chosen from the compounds of Formula A:

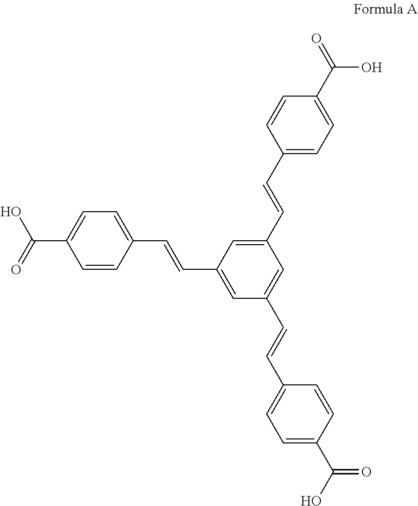 [SMILES: O=C(O)c1ccc(/C=C/c2cc(/C=C/c3ccc(C(=O)O)cc3)cc(/C=C/c3ccc(C(=O)O)cc3)c2)cc1]

or pharmaceutically acceptable salts thereof; and/or Formula B:

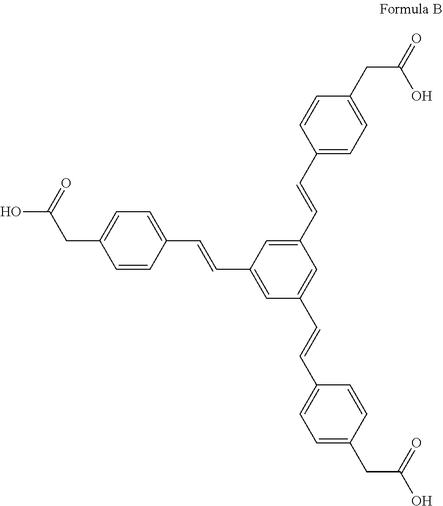 [SMILES: O=C(O)Cc1ccc(/C=C/c2cc(/C=C/c3ccc(CC(=O)O)cc3)cc(/C=C/c3ccc(CC(=O)O)cc3)c2)cc1]

or pharmaceutically acceptable salts thereof; and/or Formula C:

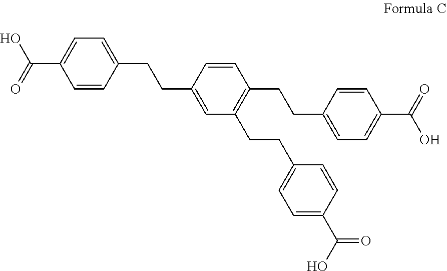 [SMILES: O=C(O)c1ccc(CCc2ccc(CCc3ccc(C(=O)O)cc3)c(CCc3ccc(C(=O)O)cc3)c2)cc1]

or pharmaceutically acceptable salts thereof; and/or Formula D:

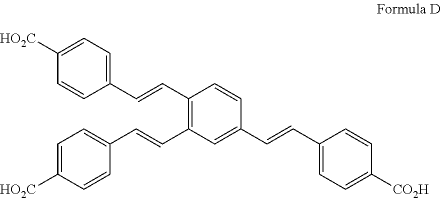 [SMILES: O=C(O)c1ccc(/C=C/c2ccc(/C=C/c3ccc(C(=O)O)cc3)c(/C=C/c3ccc(C(=O)O)cc3)c2)cc1]

or pharmaceutically acceptable salts thereof; and/or Formula E:

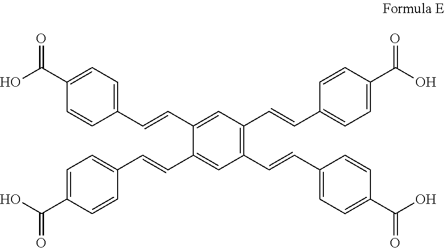 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

or pharmaceutically acceptable salts thereof.

Compounds of Formula A may alternatively be referred to by any of the following names: 1,3,5-Tris[(1E)-2′-(4″-benzoic acid)vinyl]benzene; Ramizol. The compound of Formula A will be referred to as Ramizol in the Examples provided.

Compounds of Formula B may alternatively be referred to by any of the following names: 2,2′,2″-{[(1E,1′E,1″E)-benzene-1,3,5-triyltris(ethene-2,1-diyl)]tris(benzene-4,1-diyl)}triacetic acid; Chlopam. The compound of Formula B will be referred to as Chlopam in the Examples provided.

Compounds of Formula C may alternatively be referred to by any of the following names: 1,2,4-Tris[2′-(4″-benzoic acid)ethyl]benzene. The compound of Formula C will be referred to as a compound of Formula C in the Examples provided.

Compounds of Formula D may alternatively be referred to by any of the following names: 1,2,4-Tris[2′-(4″-benzoic acid)vinyl]benzene. The compound of Formula D will be referred to as a compound of Formula D in the Examples provided.

Compounds of Formula E may alternatively be referred to by any of the following names: 1,2,4,5-Tetrakis[2′-(4″-benzoic acid)vinyl]benzene. The compound of Formula E will be referred to as a compound of Formula D in the Examples provided.

Pharmaceutically acceptable salts for the purposes of the present invention include non-toxic cation and anion salts. Examples include, but are not limited to sodium, potassium, aluminium, calcium, lithium, magnesium, zinc and from bases such as ammonium, ethylenediamine, N-methyl-glutamine, lysine, arginine, ornithine, choline, N,N′-dibenzylethlenediamine, diethylamine, piperazine, tris(hydroxymethyl)aminomethane, tetramethylammonium, acetate, lactobionate, benzenesulfonate, laurate, benzoate, malate, bicarbonate, maleate, bisulfate, mandelate, bitratrate, meyate, borate, methylbromide, bromide, methylnitrate, calcium edetate, methylsulfate, camsylate, mucate, carbonate, napsylate, chloride, nitrate, clavulanate, N-methylglucamine, citrate, hydrochloride, oleate, edetate, oxalate, edisylate, pamoate (embonate), estolate, palmitate, esylate, pantothenate, fumarate, phosphate, diphosphate, glucepate, plygalacturonate, gluconate, salicylate, glutamate, stearate, glycollylarsanilate, sulfate, hexylresorcinate, subacetate, hydrabamine, succinate, hydrobromide, tannate, tartrate, hydroxynapthoate, teoclate, iodide, tosylate, isothionate, triethiodide, lactate, panoate and valerate.

### Cephem and Cephem Derivatives

Cephem and cepham derivatives are a sub-group of β-lactam antibiotics, including cephalosporins and cephamycins. The term cephem and cepham derivatives includes the pahramceutically acceptable salts of cephem and the cepham derivatives.

Cephems are compounds of general Formula G. When V is sulphur and R₃ is a hydrogen, the cephems are called cephalosporins; and when V is sulphur and R₃ is —O—CH₃ (methoxy), the cephems are called cephamycins; when V is carbon and R₃ is hydrogen, the cephems are called carbacephems; when V is oxygen and R₃ is —O—CH₃ (methoxy), the cephems are called oxacephems.

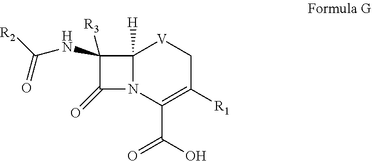

Preferably, the cephem or cephem derivative is chosen from the list comprising:
a) Cephalosporin and derivatives: Cefacetrile (cephacetrile), Cefadroxil (cefadroxyl; Duricef), Cephalexin (cefalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefclidine, cefepime (Maxipime), cefluprenam, cefoselis, cefozopran, cefpirome (Cefrom), cefquinome, cefmatilen, Ceftobiprole, ceftaroline, ceftolozane, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, cefminox, Cefotiam (Pansporin), Cefcapene, Cefdaloxime, Cefdinir (Sefdin, Zinir, Omnicef, Kefnir), Cefditoren, Cefetamet, Cefixime (Fixx, Zifi, Suprax), Cefmenoxime, Cefodizime, Cefotaxime (Claforan), Cefovecin (Convenia), Cefpimizole, Cefpodoxime (Vantin, PECEF), Cefteram, Ceftamere (Enshort), Ceftibuten (Cedax), Ceftiofur, Ceftiolene, Ceftizoxime (Cefizox), Ceftriaxone (Rocephin), Cefoperazone (Cefobid), Ceftazidime (Meezat, Fortum, Fortaz), Cefbuperazone, Cefaloram, Cefaparole, Cefcanel, Cefedrolor, Cefempidone, Cefetrizole, Cefivitril, Cefmepidium, Cefoxazole, Cefrotil, Cefsumide, Ceftioxide, Cefuracetime, and Nitrocefin;
b) Cephamycin and derivatives: Cefmetazole (Zefazone), Cefotetan (Cefotan), Cefoxitin (Mefoxin);
c) Carbacephem and derivatives: Loracarbef (Lorabid); and
d) Oxacephem and derivatives: Flomoxef, Latamoxef (moxalactam).

Pharmaceutically acceptable salts of the cepham and cepham derivatives are also provided.

More preferably, the cephem or cephem derivative chosen shows increased activity against Gram-positive bacteria relative to other cephems and cephem derivatives. For example, the cephem or cephem derivative may be chosen from the list comprising: Cefacetrile (cephacetrile), Cefadroxil (cefadroxyl; Duricef), Cephalexin (cefalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefclidine, cefepime (Maxipime), cefluprenam, cefoselis, cefozopran, cefpirome (Cefrom), cefquinome, cefmatilen, Ceftobiprole, ceftaroline, ceftolozane, flomoxef, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin. Carbacephems: loracarbef (Lorabid), cefbuperazone, cefmetazole (Zefazone), cefminox, cefotetan (Cefotan), cefoxitin (Mefoxin), Cefotiam (Pansporin), Cefcapene, Cefdaloxime, Cefdinir (Sefdin, Zinir, Omnicef, Kefnir), Cefditoren, Cefetamet, Cefixime (Fixx, Zifi, Suprax), Cefmenoxime, Cefodizime, Cefotaxime (Claforan), Cefovecin (Convenia), Cefpimizole, Cefpodoxime (Vantin, PECEF), Cefteram, Ceftamere (Enshort), Ceftibuten (Cedax), Ceftiofur, Ceftiolene, Ceftizoxime (Cefizox), Ceftriaxone (Rocephin), Cefoperazone (Cefobid), latamoxef (moxalactam) and Ceftazidime (Meezat, Fortum, Fortaz).

More preferably, the cephem or cephem derivative shows increased activity against Gram-positive Staphylococcus bacteria relative to other cephems and cephem derivatives. For example, the cephem or cephem derivative may be chosen from the list comprising: Cefacetrile (cephacetrile), Cefadroxil (cefadroxyl; Duricef), Cephalexin (cefalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefclidine, cefepime (Maxipime), cefluprenam, cefoselis, cefozopran, cefpirome (Cefrom), cefquinome, cefmatilen, Ceftobiprole, ceftaroline, ceftolozane and flomoxef.

Most preferably the cephem derivative is cefepime.

### Penicillin and Penicillin Derivatives

Penicillin and penicillin derivatives are a sub-group of β-lactam antibiotics containing a nucleus of 6-aminopenicillanic acid (lactam plus thiazolidine ring) and other ring side-chains. Penicillins are compounds of general Formula H, with different R groups representing different antibiotics. For example, if the R group is a dimethoxybenzene it is methicillin, if the R group is a benzyl it is penicillin G. The term penicillin or penicillin derivative includes the pharmaceutically acceptable salts of penicillin and the penicillin derivatives.

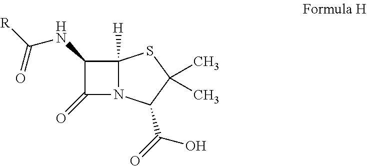

Preferably, the penicillin or penicillin derivative is chosen from the list comprising: penicillin G, penicillin V, methicillin, nafcillin, oxacillin, cloxacillin, dicloxacillin, ampicillin, amoxicillin, carbenicillin, ticarcillin, mezlocillin, and piperacillin.

More preferably the penicillin or penicillin derivative is a β-lactamase-resistant penicillin or penicillin derivative. Preferably the penicillin or penicillin derivative is methicillin, nafcillin, oxacillin, cloxacillin, or dicloxacillin.

Most preferably the penicillin derivative is oxacillin.

The present invention provides a composition comprising a combination of: (i) at least one aryl antibiotic of Group I or pharmaceutically acceptable salts thereof; and (ii) cefepime and/or oxacillin.

Vancomycin and daptomycin (macrolide antibiotics), linezolid (oxazolidinone antibiotic), and mupirocin (monoxycarbolic acid antibiotic) are antibiotics often used for the treatment of infections caused by multi-resistant bacteria including Streptococcus and Staphylococcus strains, particularly methicillin-resistant Staphylococcus aureus (MRSA). Also included are the pharmaceutically acceptable salts of vancomycin, linezolid, daptomycin and mupirocin.

The pharmaceutically acceptable salts of the penicillin or penicillin derivatives, cephem and cepham derivatives, vancomycin, linezolid, daptomycin and mupirocin include the non-toxic cation and anion salts. Examples include, but are not limited to sodium, potassium, aluminium, calcium, lithium, magnesium, zinc and from bases such as ammonium, ethylenediamine, N-methyl-glutamine, lysine, arginine, ornithine, choline, N,N′-dibenzylethlenediamine, diethylamine, piperazine, tris(hydroxymethyl)aminomethane, tetramethylammonium, acetate, lactobionate, benzenesulfonate, laurate, benzoate, malate, bicarbonate, maleate, bisulfate, mandelate, bitratrate, meyate, borate, methylbromide, bromide, methylnitrate, calcium edetate, methylsulfate, camsylate, mucate, carbonate, napsylate, chloride, nitrate, clavulanate, N-methylglucamine, citrate, hydrochloride, oleate, edetate, oxalate, edisylate, pamoate (embonate), estolate, palmitate, esylate, pantothenate, fumarate, phosphate, diphosphate, glucepate, plygalacturonate, gluconate, salicylate, glutamate, stearate, glycollylarsanilate, sulfate, hexylresorcinate, subacetate, hydrabamine, succinate, hydrobromide, tannate, tartrate, hydroxynapthoate, teoclate, iodide, tosylate, isothionate, triethiodide, lactate, panoate and valerate salts.

Preferably, the present invention provides:
a composition comprising a combination of: (i) at least one aryl antibiotic of Group I or pharmaceutically acceptable salts thereof; and (ii) oxacillin;
a composition comprising a combination of: (i) at least one aryl antibiotic of Group I or pharmaceutically acceptable salts thereof; and (ii) cefepime.

Preferably, the present invention provides:
a composition comprising a combination of: (i) at least one aryl antibiotic of Formula A or Formula B or pharmaceutically acceptable salts thereof; and cefepime and/or oxacillin;
a composition comprising a combination of: (i) at least one aryl antibiotic of Formula A or Formula B or pharmaceutically acceptable salts thereof; and (ii) oxacillin;
a composition comprising a combination of: (i) at least one aryl antibiotic of Formula A or Formula B or pharmaceutically acceptable salts thereof; and (ii) cefepime;
a composition comprising a combination of: (i) an aryl antibiotic of Formula A or pharmaceutically acceptable salts thereof; and (ii) cefepime and/or oxacillin;
a composition comprising a combination of: (i) an aryl antibiotic of Formula B or pharmaceutically acceptable salts thereof; and (ii) cefepime and/or oxacillin;
a composition comprising a combination of: (i) an aryl antibiotic of Formula A or pharmaceutically acceptable salts thereof; and (ii) oxacillin;
a composition comprising a combination of: (i) an aryl antibiotic of Formula A or pharmaceutically acceptable salts thereof; and (ii) cefepime;
a pharmaceutical composition comprising a combination of: (i) an aryl antibiotic of Formula B or pharmaceutically acceptable salts thereof; and (ii) oxacillin; and/or
a composition comprising a combination of: (i) an aryl antibiotic of Formula B or pharmaceutically acceptable salts thereof; and (ii) cefepime.

In one form of the invention, the antibiotic combination of Chlopam and Vancomycin is used as follows: 0.003901 μg/mL and 1 μg/mL, 0.0078125 and 1 μg/mL, 0.015625 μg/mL and 1 μg/mL, 0.125 μg/mL and 0.5 μg/mL, and 0.25 μg/mL and 0.5 μg/mL. In one form of the invention, the antibiotic combination of Chlopam and Vancomycin is used at these ratios.

In one form of the invention, the antibiotic combination of Chlopam and Vancomycin is used as follows: 0.001953 μg/mL and 1 μg/mL, 0.003901 μg/mL and 1 μg/mL, 0.0078125 and 1 μg/mL, and 0.015625 μg/mL and 1 μg/mL In one form of the invention, the antibiotic combination of Chlopam and Vancomycin is used at these ratios.

In one form of the invention, the antibiotic combination of Chlopam and Linezolid is used as follows: 0.003901 μg/mL and 2 μg/mL, 0.0078125 and 2 μg/mL, 0.015625 μg/mL and 2 μg/mL, 0.125 μg/mL and 1 μg/mL, 0.25 μg/mL and 0.5 μg/mL, and 0.5 μg/mL and 0.25 μg/mL. In one form of the invention, the antibiotic combination of Chlopam and Linezolid is used at these ratios.

In one form of the invention, the antibiotic combination of Chlopam and Linezolid is used as follows: 0.001953 μg/mL and 1 μg/mL, 0.003901 μg/mL and 1 μg/mL, 0.0078125 and 1 μg/mL, and 0.015625 μg/mL and 1 μg/mL In one form of the invention, the antibiotic combination of Chlopam and Linezolid is used at these ratios.

In one form of the invention, the antibiotic combination of Chlopam and Daptomycin is used as follows: 0.003901 μg/mL and 0.5 μg/mL, 0.0078125 and 0.5 μg/mL, 0.015625 μg/mL and 0.5 μg/mL, 0.25 μg/mL and 0.25 μg/mL, and 0.5 μg/mL and 0.016 μg/mL. In one form of the invention, the antibiotic combination of Chlopam and Daptomycin is used at these ratios.

In one form of the invention, the antibiotic combination of Chlopam and Daptomycin is used as follows: 0.001953 μg/mL and 0.5 μg/mL, 0.003901 μg/mL and 0.5 μg/mL, 0.0078125 and 0.5 μg/mL, and 0.015625 μg/mL and 0.5 μg/mL, and 0.5 μg/mL and 0.016 μg/mL. In one form of the invention, the antibiotic combination of Chlopam and Daptomycin is used at these ratios.

In one form of the invention, the antibiotic combination of Chlopam and Mupirocin is used as follows: 0.003901 μg/mL and 0.5 μg/mL, 0.0078125 and 0.5 μg/mL, 0.015625 μg/mL and 0.5 μg/mL, and 0.25 μg/mL and 0.25 μg/mL. In one form of the invention, the antibiotic combination of Chlopam and Mupirocin is used at these ratios.

In one form of the invention, the antibiotic combination of Chlopam and Mupirocin is used as follows: 0.001953 μg/mL and 0.5 μg/mL, 0.003901 μg/mL and 0.5 μg/mL, 0.0078125 and 0.5 μg/mL, and 0.015625 μg/mL and 0.5 μg/mL, and 0.5 μg/mL and 0.016 μg/mL. In one form of the invention, the antibiotic combination of Chlopam and Mupirocin is used at these ratios.

In one form of the invention, the antibiotic combination of Ramizol and Vancomycin is used as follows: 0.015625 μg/mL and 1 μg/mL, 0.03125 μg/mL and 1 μg/mL, 0.0625 and 1 μg/mL, and 0.125 μg/mL and 1 μg/mL. In one form of the invention, the antibiotic combination of Ramizol and Vancomycin is used at these ratios.

In one form of the invention, the antibiotic combination of Ramizol and Vancomycin is used as follows: 0.015625 μg/mL and 1 μg/mL, 0.03125 μg/mL and 1 μg/mL, 0.0625 and 1 μg/mL, 0.125 μg/mL and 1 μg/mL, and 4 μg/mL and 0.031 μg/mL. In one form of the invention, the antibiotic combination of Ramizol and Vancomycin is used at these ratios.

In one form of the invention, the antibiotic combination of Ramizol and Linezolid is used as follows: 0.015625 μg/mL and 4 μg/mL, 0.03125 μg/mL and 4 μg/mL, 0.0625 and 4 μg/mL, 0.125 μg/mL and 4 μg/mL, and 0.25 μg/mL and 4 μg/mL. In one form of the invention, the antibiotic combination of Ramizol and Linezolid is used at these ratios.

In one form of the invention, the antibiotic combination of Ramizol and Linezolid is used as follows: 0.015625 μg/mL and 1 μg/mL, 0.03125 μg/mL and 1 μg/mL, 0.0625 and 1 μg/mL, and 0.125 μg/mL and 1 μg/mL. In one form of the invention, the antibiotic combination of Ramizol and Linezolid is used at these ratios.

In one form of the invention, the antibiotic combination of Ramizol and Daptomycin is used as follows: 0.015625 μg/mL and 0.5 μg/mL, 0.03125 μg/mL and 0.5 μg/mL, 0.0625 and 0.5 μg/mL, 0.125 μg/mL and 0.5 μg/mL, 2 μg/mL and 0.25 μg/mL, 2 μg/mL and 0.125 μg/mL, 2 μg/mL and 0.063 μg/mL, 2 μg/mL and 0.031 μg/mL, and 4 μg/mL and 0.016 μg/mL. In one form of the invention, the antibiotic combination of Ramizol and Daptomycin is used at these ratios.

In one form of the invention, the antibiotic combination of Ramizol and Daptomycin is used as follows: 0.015625 μg/mL and 0.5 μg/mL, 0.03125 μg/mL and 0.5 μg/mL, 0.0625 and 0.5 μg/mL, 0.125 μg/mL and 0.5 μg/mL, 2 μg/mL and 0.25 μg/mL, and 4 μg/mL and 0.016 μg/mL. In one form of the invention, the antibiotic combination of Ramizol and Daptomycin is used at these ratios.

In one form of the invention, the antibiotic combination of Ramizol and Mupirocin is used as follows: 0.015625 μg/mL and 0.5 μg/mL, 0.03125 μg/mL and 0.5 μg/mL, 0.0625 and 0.5 μg/mL, 0.125 μg/mL and 0.5 μg/mL, and 0.25 μg/mL and 0.5 μg/mL. In one form of the invention, the antibiotic combination of Ramizol and Mupirocin is used at these ratios.

In one form of the invention, the antibiotic combination of Ramizol and Mupirocin is used as follows: 0.015625 μg/mL and 0.5 μg/mL, 0.03125 μg/mL and 0.5 μg/mL, 0.0625 and 0.5 μg/mL, 0.125 μg/mL and 0.5 μg/mL, and 4 μg/mL and 0.016 μg/mL. In one form of the invention, the antibiotic combination of Ramizol and Mupirocin is used at these ratios.

In one form of the invention, the antibiotic combination of Chlopam and Oxacillin is used as follows: 0.0125 μg/mL and 0.125 μg/mL. In one form of the invention, the antibiotic combination of Chlopam and Oxacillin is used at these ratios.

In one form of the invention, the antibiotic combination of Chlopam and Cefepime is used as follows: 0.25 μg/mL and 64 μg/mL, 0.25 μg/mL and 32 μg/mL, 0.25 μg/mL and 16 μg/mL, and 0.50 μg/mL and 8 μg/mL. In one form of the invention, the antibiotic combination of Chlopam and Cefepime is used at these ratios.

In one form of the invention, the antibiotic combination of Ramizol and Cefepime is used as follows: 0.125 μg/mL and 128 μg/mL, 1 μg/mL and 64 μg/mL, and 2 μg/mL and 8 μg/mL. In one form of the invention, the antibiotic combination of Ramizol and Cefepime is used at these ratios.

Aspects of the invention also relate to antibacterial methods. Preferably, the compositions according to the present invention are active against a variety of bacterial organisms, in particular against Gram-positive bacteria.

In one embodiment, aspects of the invention include methods for preventing bacterial growth. In another embodiment, aspects of the invention include methods for stopping bacterial growth. In a further embodiment, aspects of the invention include methods for killing bacterial cells. Methods of the invention can be performed in vivo, ex vivo, in vitro, etc. Methods of the invention may be particularly useful to kill or inhibit (e.g., to prevent or stop the growth of) drug-resistant bacterial cells (e.g., antibiotic-resistant bacterial cells). Methods and compositions of the invention may be particularly useful for killing or inhibiting drug-resistant bacterial cells using combination doses, particularly low doses of at least one aryl antibiotic of Group I or pharmaceutically acceptable salts thereof. In other embodiments, combinations of the invention may be useful for killing or inhibiting drug-resistant bacterial cells using low doses of at least one antibiotic selected from the list comprising a penicillin or a derivative thereof, a cepham or a derivative thereof, vancomycin, linezolid, daptomycin and mupirocin.

The compositions can be used in a method for suppressing, inhibiting, preventing, alleviating or treating infectious diseases including e.g. nosocomial infections, community-acquired infections, skin infections (eg impetigo and/or cellulitis), pneumonia, food poisoning, toxic shock syndrome, blood poisoning (bacteremia) and sepsis caused by bacterial organisms. The bacterial organisms may preferably be antibiotic resistant bacteria.

The present invention provides a method for suppressing, inhibiting, preventing, alleviating or treating a bacterial infection, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.
Preferably the antibiotic of (ii) is a penicillin or a derivative thereof or a cepham or a derivative thereof.

The present invention provides a method for suppressing, inhibiting, preventing, alleviating or treating a bacterial infection, the method comprising the step of:
administering a composition comprising a combination of: (i) at least one aryl antibiotic of Group I or pharmaceutically acceptable salts thereof; and (ii) a penicillin or a derivative thereof;
administering a composition comprising a combination of: (i) at least one aryl antibiotic of Group I or pharmaceutically acceptable salts thereof; and (ii) cepham or a derivative thereof;
administering a composition comprising a combination of: (i) at least one aryl antibiotic of Group I or pharmaceutically acceptable salts thereof; and (ii) vancomycin;
administering a composition comprising a combination of: (i) at least one aryl antibiotic of Group I or pharmaceutically acceptable salts thereof; and (ii) linezolid;
administering a composition comprising a combination of: (i) at least one aryl antibiotic of Group I or pharmaceutically acceptable salts thereof; and (ii) daptomycin; and/or
administering a composition comprising a combination of: (i) at least one aryl antibiotic of Group I or pharmaceutically acceptable salts thereof; and (ii) mupirocin.

The present invention provides a method for suppressing, inhibiting, preventing, alleviating or treating a bacterial infection, the method comprising the step of:
administering a composition comprising a combination of: (i) at least one aryl antibiotic of Formula A or Formula B or pharmaceutically acceptable salts thereof; and (ii) at least one antibiotic selected from the list comprising a penicillin or a derivative thereof or cepham or a derivative thereof;
administering a composition comprising a combination of: (i) at least one aryl antibiotic of Formula A or Formula B or pharmaceutically acceptable salts thereof; and (ii) a penicillin or a derivative thereof;
administering a composition comprising a combination of: (i) at least one aryl antibiotic of Formula A or Formula B or pharmaceutically acceptable salts thereof; and (ii) cepham or a derivative thereof;
administering a composition comprising a combination of: (i) at least one aryl antibiotic of Formula A or Formula B or pharmaceutically acceptable salts thereof; and (ii) vancomycin;
administering a composition comprising a combination of: (i) at least one aryl antibiotic of Formula A or Formula B or pharmaceutically acceptable salts thereof; and (ii) linezolid;
administering a composition comprising a combination of: (i) at least one aryl antibiotic of Formula A or Formula B or pharmaceutically acceptable salts thereof; and (ii) daptomycin;
administering a composition comprising a combination of: (i) at least one aryl antibiotic of Formula A or Formula B or pharmaceutically acceptable salts thereof; and (ii) mupirocin;
administering a composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula A or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula B or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula A or pharmaceutically acceptable salts thereof; and (ii) at least one penicillin or a derivative thereof;
administering a composition comprising a combination of: (i) an aryl antibiotic of Formula A or pharmaceutically acceptable salts thereof; and (ii) at least one cepham or a derivative thereof;
administering a composition comprising a combination of: (i) an aryl antibiotic of Formula A or pharmaceutically acceptable salts thereof; and (ii) vancomycin;
administering a composition comprising a combination of: (i) an aryl antibiotic of Formula A or pharmaceutically acceptable salts thereof; and (ii) linezolid;
administering a composition comprising a combination of: (i) an aryl antibiotic of Formula A or pharmaceutically acceptable salts thereof; and (ii) daptomycin;
administering a composition comprising a combination of: (i) an aryl antibiotic of Formula A or pharmaceutically acceptable salts thereof; and (ii) mupirocin;
administering a pharmaceutical composition comprising a combination of: (i) an aryl antibiotic of Formula B or pharmaceutically acceptable salts thereof; and (ii) at least one penicillin or a derivative thereof;
administering a composition comprising a combination of: (i) an aryl antibiotic of Formula B or pharmaceutically acceptable salts thereof; and (ii) at least one cepham or a derivative thereof;
administering a composition comprising a combination of: (i) an aryl antibiotic of Formula B or pharmaceutically acceptable salts thereof; and (ii) vancomycin;
administering a composition comprising a combination of: (i) an aryl antibiotic of Formula B or pharmaceutically acceptable salts thereof; and (ii) linezolid;
administering a composition comprising a combination of: (i) an aryl antibiotic of Formula B or pharmaceutically acceptable salts thereof; and (ii) daptomycin; and/or
administering a composition comprising a combination of: (i) an aryl antibiotic of Formula B or pharmaceutically acceptable salts thereof; and (ii) mupirocin.

The compositions may further or additionally be used in the control or manipulation of commensal bacterial populations in subjects that do not have an infection, but that carry a normal microbial population that includes bacteria that may become problematic and cause infections after the surgery or immunocompromising event. For example, the compositions may be used to control or manipulate a bacterial population in subject that is about to undergo surgery, or become immunocompromised through, for example, chemotherapy. It may be advantageous to eliminate or at least reduce the numbers of one or more bacterial species that form the normal body flora of the subject before the surgery or immunocompromising event.

The present invention provides a method to control or manipulate a commensal bacterial population, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.
   Preferably the antibiotic of (ii) is a penicillin or a derivative thereof or a cepham or a derivative thereof.

The present invention provides a method to control or manipulate a bacterial population, the method comprising the step of:
administering a composition comprising a combination of: (i) at least one aryl antibiotic of Group I or pharmaceutically acceptable salts thereof; and (ii) a penicillin or a derivative thereof;
administering a composition comprising a combination of: (i) at least one aryl antibiotic of Group I or pharmaceutically acceptable salts thereof; and (ii) a cepham or a derivative thereof;
administering a composition comprising a combination of: (i) at least one aryl antibiotic of Group I or pharmaceutically acceptable salts thereof; and (ii) vancomycin;
administering a composition comprising a combination of: (i) at least one aryl antibiotic of Group I or pharmaceutically acceptable salts thereof; and (ii) linezolid;
administering a composition comprising a combination of: (i) at least one aryl antibiotic of Group I or pharmaceutically acceptable salts thereof; and (ii) daptomycin; and/or
administering a composition comprising a combination of: (i) at least one aryl antibiotic of Group I or pharmaceutically acceptable salts thereof; and (ii) mupirocin.

The present invention provides a method to control or manipulate a bacterial population, the method comprising the step of:
administering a composition comprising (i) at least one aryl antibiotic of Formula A or Formula B or pharmaceutically acceptable salts thereof and (ii) at least one antibiotic selected from the list comprising penicillin or a derivative thereof or cepham or a derivative thereof;
administering a composition comprising a combination of: (i) at least one aryl antibiotic of Formula A or Formula B or pharmaceutically acceptable salts thereof; and (ii) a penicillin or a derivative thereof;
administering a composition comprising a combination of: (i) at least one aryl antibiotic of Formula A or Formula B or pharmaceutically acceptable salts thereof; and (ii) a cepham or a derivative thereof;
administering a composition comprising a combination of: (i) at least one aryl antibiotic of Formula A or Formula B or pharmaceutically acceptable salts thereof; and (ii) vancomycin;
administering a composition comprising a combination of: (i) at least one aryl antibiotic of Formula A or Formula B or pharmaceutically acceptable salts thereof; and (ii) linezolid;
administering a composition comprising a combination of: (i) at least one aryl antibiotic of Formula A or Formula B or pharmaceutically acceptable salts thereof; and (ii) daptomycin;
administering a composition comprising a combination of: (i) at least one aryl antibiotic of Formula A or Formula B or pharmaceutically acceptable salts thereof; and (ii) mupirocin;
administering a composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof;
administering a composition comprising a combination of: (i) an aryl antibiotic of Formula A or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof;
administering a composition comprising a combination of: (i) an aryl antibiotic of Formula B or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula A or pharmaceutically acceptable salts thereof; and (ii) at least one penicillin or a derivative thereof;
administering a composition comprising a combination of: (i) an aryl antibiotic of Formula A or pharmaceutically acceptable salts thereof; and (ii) at least one cepham or a derivative thereof;
administering a composition comprising a combination of: (i) an aryl antibiotic of Formula A or pharmaceutically acceptable salts thereof; and (ii) vancomycin;
administering a composition comprising a combination of: (i) an aryl antibiotic of Formula A or pharmaceutically acceptable salts thereof; and (ii) linezolid;
administering a composition comprising a combination of: (i) an aryl antibiotic of Formula A or pharmaceutically acceptable salts thereof; and (ii) daptomycin;
administering a composition comprising a combination of: (i) an aryl antibiotic of Formula A or pharmaceutically acceptable salts thereof; and (ii) mupirocin;
administering a composition comprising a combination of: (i) an aryl antibiotic of Formula B or pharmaceutically acceptable salts thereof; and (ii) at least one penicillin or a derivative thereof;
administering a composition comprising a combination of: (i) an aryl antibiotic of Formula B or pharmaceutically acceptable salts thereof; and (ii) at least one cepham or a derivative thereof
administering a composition comprising a combination of: (i) an aryl antibiotic of Formula B or pharmaceutically acceptable salts thereof; and (ii) vancomycin;
administering a composition comprising a combination of: (i) an aryl antibiotic of Formula B or pharmaceutically acceptable salts thereof; and (ii) linezolid;
administering a composition comprising a combination of: (i) an aryl antibiotic of Formula B or pharmaceutically acceptable salts thereof; and (ii) daptomycin; and/or
administering a composition comprising a combination of: (i) an aryl antibiotic of Formula B or pharmaceutically acceptable salts thereof; and (ii) mupirocin.

The compositions of present invention may be used in methods to delay or prevent the development of antibiotic resistance in bacteria. The antibiotic resistance may be antibiotic resistance that develops within the bacterial population infecting a subject. Alternatively, the antibiotic resistance may develop in the bacterial population present in an environmental contamination setting, such as in or on devices and/or compositions (such as medical and/or dental equipment, devices, and/or compositions) and/or facilities at medical and/or dental centers (e.g., hospital rooms, operating rooms, emergency rooms, etc). In either an infective or a contamination setting, the avoidance of the development of antibiotic resistance may be increased (either in time or in strength) by use of the method of the present invention. For example, the method of the present invention may delay the development of antibiotic resistance for a period of time, and/or the method of the present invention may reduce the strength of the antibiotic resistance, such that the antibiotics are still effective, albeit at a higher dose than was previously effective.

The present invention provides a method to delay or prevent the development of antibiotic resistance in a bacteria to the aryl antibiotics of Formula A or Formula B or pharmaceutically acceptable salts thereof and/or the antibiotics selected from the list comprising a penicillin or a derivative thereof, a cepham or a derivative thereof, vancomycin, linezolid, daptomycin and mupirocin, the method comprising the step of:
a) administering a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.
   Preferably the antibiotic of (ii) is a penicillin or a derivative thereof or a cepham or a derivative thereof.

The present invention provides a method to delay or prevent the development of antibiotic resistance in a bacteria to the aryl antibiotics of Group I or pharmaceutically acceptable salts thereof and/or the antibiotics selected from the list comprising penicillin or a derivative thereof and/or cepham or a derivative thereof, the method comprising the step of:
administering a composition comprising a combination of: (i) at least one aryl antibiotic of Group I or pharmaceutically acceptable salts thereof; and (ii) a penicillin or a derivative thereof;
administering a composition comprising a combination of: (i) at least one aryl antibiotic of Group I or pharmaceutically acceptable salts thereof; and (ii) a cepham or a derivative thereof;
administering a composition comprising a combination of: (i) at least one aryl antibiotic of Group I or pharmaceutically acceptable salts thereof; and (ii) vancomycin;
administering a composition comprising a combination of: (i) at least one aryl antibiotic of Group I or pharmaceutically acceptable salts thereof; and (ii) linezolid;
administering a composition comprising a combination of: (i) at least one aryl antibiotic of Group I or pharmaceutically acceptable salts thereof; and (ii) daptomycin; and/or
administering a composition comprising a combination of: (i) at least one aryl antibiotic of Group I or pharmaceutically acceptable salts thereof; and (ii) mupirocin.

The present invention provides a method to delay or prevent the development of antibiotic resistance in a bacteria to the aryl antibiotics of Formula A and/or Formula B or pharmaceutically acceptable salts thereof and/or the antibiotics selected from the list comprising penicillin or a derivative thereof and/or cepham or a derivative thereof, the method comprising the step of:
administering a composition comprising a combination of: (i) at least one aryl antibiotic of Formula A or Formula B or pharmaceutically acceptable salts thereof; and (ii) at least one antibiotic selected from the list comprising a penicillin or a derivative thereof or cepham or a derivative thereof;
administering a composition comprising a combination of: (i) at least one aryl antibiotic of Formula A or Formula B or pharmaceutically acceptable salts thereof; and (ii) a penicillin or a derivative thereof;
administering a composition comprising a combination of: (i) at least one aryl antibiotic of Formula A or Formula B or pharmaceutically acceptable salts thereof; and (ii) ar cepham or a derivative thereof;
administering a composition comprising a combination of: (i) at least one aryl antibiotic of Formula A or Formula B or pharmaceutically acceptable salts thereof; and (ii) vancomycin;
administering a composition comprising a combination of: (i) at least one aryl antibiotic of Formula A or Formula B or pharmaceutically acceptable salts thereof; and (ii) linezolid;
administering a composition comprising a combination of: (i) at least one aryl antibiotic of Formula A or Formula B or pharmaceutically acceptable salts thereof; and (ii) daptomycin;
administering a composition comprising a combination of: (i) at least one aryl antibiotic of Formula A or Formula B or pharmaceutically acceptable salts thereof; and (ii) mupirocin;
administering a composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula A or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof;
a composition comprising a combination of: (i) an aryl antibiotic of Formula B or pharmaceutically acceptable salts thereof; and (ii) at least one antibiotic selected from the list comprising vancomycin, linezolid, daptomycin and mupirocin;
administering a composition comprising a combination of: (i) an aryl antibiotic of Formula A or pharmaceutically acceptable salts thereof; and (ii) at least one penicillin or a derivative thereof;
administering a composition comprising a combination of: (i) an aryl antibiotic of Formula A or pharmaceutically acceptable salts thereof; and (ii) at least one cepham or a derivative thereof;
administering a composition comprising a combination of: (i) an aryl antibiotic of Formula A or pharmaceutically acceptable salts thereof; and (ii) vancomycin;
administering a composition comprising a combination of: (i) an aryl antibiotic of Formula A or pharmaceutically acceptable salts thereof; and (ii) linezolid;
administering a composition comprising a combination of: (i) an aryl antibiotic of Formula A or pharmaceutically acceptable salts thereof; and (ii) daptomycin;
administering a composition comprising a combination of: (i) an aryl antibiotic of Formula A or pharmaceutically acceptable salts thereof; and (ii) mupirocin;
administering a pharmaceutical composition comprising a combination of: (i) an aryl antibiotic of Formula B or pharmaceutically acceptable salts thereof; and (ii) at least one penicillin or a derivative thereof;
administering a composition comprising a combination of: (i) an aryl antibiotic of Formula B or pharmaceutically acceptable salts thereof; and (ii) at least one cepham or a derivative thereof.
administering a composition comprising a combination of: (i) an aryl antibiotic of Formula B or pharmaceutically acceptable salts thereof; and (ii) vancomycin;
administering a composition comprising a combination of: (i) an aryl antibiotic of Formula B or pharmaceutically acceptable salts thereof; and (ii) linezolid;
administering a composition comprising a combination of: (i) an aryl antibiotic of Formula B or pharmaceutically acceptable salts thereof; and (ii) daptomycin; and/or
administering a composition comprising a combination of: (i) an aryl antibiotic of Formula B or pharmaceutically acceptable salts thereof; and (ii) mupirocin.

Aspects of the invention also relate to sterilizing devices and/or compositions to prevent, remove or control bacterial contamination. In one embodiment, the method may be used to prevent, remove or control bacterial contamination on medical and/or dental equipment, devices, and/or compositions. In another embodiment, may be used to prevent, remove or control bacterial contamination on facilities at medical and/or dental centers (e.g., hospital rooms, operating rooms, emergency rooms, etc.).

The present invention provides a method to prevent, remove or control bacterial contamination, the method comprising the step of:
a) applying a composition comprising (i) at least one aryl antibiotic of Group I or pharmaceutically acceptable salts thereof and (ii) at least one antibiotic selected from the list comprising a penicillin or a derivative thereof, a cepham or a derivative thereof, vancomycin, linezolid, daptomycin and mupirocin.
Preferably the antibiotic of (ii) is a penicillin or a derivative thereof or a cepham or a derivative thereof.

The present invention provides a method to prevent, remove or control bacterial contamination, the method comprising the step of:
applying a composition comprising a combination of: (i) at least one aryl antibiotic of Group I or pharmaceutically acceptable salts thereof; and (ii) a penicillin or a derivative thereof;
applying a composition comprising a combination of: (i) at least one aryl antibiotic of Group I or pharmaceutically acceptable salts thereof; and (ii) ar cepham or a derivative thereof;
applying a composition comprising a combination of: (i) at least one aryl antibiotic of Group I or pharmaceutically acceptable salts thereof; and (ii) vancomycin;
applying a composition comprising a combination of: (i) at least one aryl antibiotic of Group I or pharmaceutically acceptable salts thereof; and (ii) linezolid;
applying a composition comprising a combination of: (i) at least one aryl antibiotic of Group I or pharmaceutically acceptable salts thereof; and (ii) daptomycin; and/or
applying a composition comprising a combination of: (i) at least one aryl antibiotic of Group I or pharmaceutically acceptable salts thereof; and (ii) mupirocin.

The present invention provides a method to prevent, remove or control bacterial contamination, the method comprising the step of:
applying a composition comprising a combination of: (i) at least one aryl antibiotic of Formula A or Formula B or pharmaceutically acceptable salts thereof; and (ii) at least one antibiotic selected from the list comprising penicillin or a derivative thereof or cepham or a derivative thereof;
applying a composition comprising a combination of: (i) at least one aryl antibiotic of Formula A or Formula B or pharmaceutically acceptable salts thereof; and (ii) a penicillin or a derivative thereof;
applying a composition comprising a combination of: (i) at least one aryl antibiotic of Formula A or Formula B or pharmaceutically acceptable salts thereof; and (ii) ar cepham or a derivative thereof;
applying a composition comprising a combination of: (i) at least one aryl antibiotic of Formula A or Formula B or pharmaceutically acceptable salts thereof; and (ii) vancomycin;
applying a composition comprising a combination of: (i) at least one aryl antibiotic of Formula A or Formula B or pharmaceutically acceptable salts thereof; and (ii) linezolid;
applying a composition comprising a combination of: (i) at least one aryl antibiotic of Formula A or Formula B or pharmaceutically acceptable salts thereof; and (ii) daptomycin;
applying a composition comprising a combination of: (i) at least one aryl antibiotic of Formula A or Formula B or pharmaceutically acceptable salts thereof; and (ii) mupirocin;
applying a composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof;
applying a composition comprising a combination of: (i) an aryl antibiotic of Formula A or pharmaceutically acceptable salts thereof; and (ii) at least one antibiotic selected from the list comprising vancomycin, linezolid, daptomycin and mupirocin;
applying a composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof;
applying a composition comprising a combination of: (i) an aryl antibiotic of Formula B or pharmaceutically acceptable salts thereof; and (ii) at least one antibiotic selected from the list comprising vancomycin, linezolid, daptomycin and mupirocin;
applying a composition comprising a combination of: (i) an aryl antibiotic of Formula A or pharmaceutically acceptable salts thereof; and (ii) at least one penicillin or a derivative thereof;
applying a composition comprising a combination of: (i) an aryl antibiotic of Formula A or pharmaceutically acceptable salts thereof; and (ii) at least one cepham or a derivative thereof;
applying a composition comprising a combination of: (i) an aryl antibiotic of Formula A or pharmaceutically acceptable salts thereof; and (ii) vancomycin;
applying a composition comprising a combination of: (i) an aryl antibiotic of Formula A or pharmaceutically acceptable salts thereof; and (ii) linezolid;
applying a composition comprising a combination of: (i) an aryl antibiotic of Formula A or pharmaceutically acceptable salts thereof; and (ii) daptomycin;
applying a composition comprising a combination of: (i) an aryl antibiotic of Formula A or pharmaceutically acceptable salts thereof; and (ii) mupirocin;
applying a composition comprising a combination of: (i) an aryl antibiotic of Formula B or pharmaceutically acceptable salts thereof; and (ii) at least one penicillin or a derivative thereof;
applying a composition comprising a combination of: (i) an aryl antibiotic of Formula B or pharmaceutically acceptable salts thereof; and (ii) at least one cepham or a derivative thereof;
applying a composition comprising a combination of: (i) an aryl antibiotic of Formula B or pharmaceutically acceptable salts thereof; and (ii) vancomycin;
applying a composition comprising a combination of: (i) an aryl antibiotic of Formula B or pharmaceutically acceptable salts thereof; and (ii) linezolid;
applying a composition comprising a combination of: (i) an aryl antibiotic of Formula B or pharmaceutically acceptable salts thereof; and (ii) daptomycin; and/or
applying a composition comprising a combination of: (i) an aryl antibiotic of Formula B or pharmaceutically acceptable salts thereof; and (ii) mupirocin.

Preferably, the compositions comprising a combination of: (i) at least one aryl antibiotic of Formula A or Formula B or pharmaceutically acceptable salts thereof; and (ii) at least one antibiotic selected from the list comprising penicillin or a derivative thereof or cepham or a derivative thereof are active against a Staphylococcus species.

The compositions and methods of the present invention are therefore used in the methods provided to:
suppress, inhibit, prevent, alleviate or treat a bacterial infection;
control or manipulate a commensal bacterial population;
delay or prevent the development of antibiotic resistance in a bacteria to the aryl antibiotics of Group I or pharmaceutically acceptable salts thereof;
delay or prevent the development of antibiotic resistance in a bacteria to the antibiotics selected from the list comprising penicillin or a derivative thereof or cepham or a derivative thereof; and/or
prevent, remove or control bacterial contamination
   wherein the bacteria to be suppressed, inhibited, prevented, alleviated or treated; controlled or manipulated; prevented or delayed from developing antibiotic resistance; or the bacterial contamination prevented, removed or controlled is a Staphylococcus species.

The bacteria to be suppressed, inhibited, prevented, alleviated or treated; controlled or manipulated; prevented or delayed from developing antibiotic resistance; or the bacterial contamination prevented, removed or controlled may be a single species of Staphylococcus, or may be a mixture of two or more species.

The Staphylococcus species may be chosen from the list comprising: S. arlettae, S. agnetis, S. aureus, S. auricularis, S. capitis, S. caprae, S. carnosus, S. caseolyticus, S. chromogenes, S. cohnii, S. condimenti, S. croceolyticus, S. delphini, S. devriesei, S. epidermidis, S. equorum, S. faecalis, S. fells, S. fleurettii, S. gallinarum, S. haemolyticus, S. hominis, S. hyicus, S. intermedius, S. kloosii, S. leei, S. lentus, S. lugdunensis, S. lutrae, S. lyticans, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. roseus, S. saccharolyticus, S. saprophyticus, S. schleiferi, S. sciuri, S. simiae, S. simulans, S. stepanovicii, S. succinus, S. vitulinus, S. warneri, and S. xylosus.

More preferably, the Staphylococcus species is a species that can infect humans. For example, the species may be chosen from the list comprising: S. aureus, S. auricularis, S.carnosus, S. capitis, S. caprae, S. cohnii, S. epidermidis, S. haemolyticus, S.hyicus, S. leei, S. lugdunensis, S. pasteuri, S. pettenkoferi, S.saprophyticus, S. schleiferi, S. sciuri, S. simulans, S. warneri, and S. xylosus. More preferably, the species may be chosen from the list comprising: S.aureus, S.auricularis, S.carnosus, S.epidermidis, S.haemolyticus, S.hyicus, S.lugdunensis, S.saprophyticus, S.sciuri, S.simulans and S.warneri.

Alternatively, the Staphylococcus species may be a species that can infect an animal of economic, agricultural or social importance. For example, the following Staphylococcus species are thought to infect animals of economic, agricultural or social importance: S. arlattae—chickens, goats; S. aureus—cattle; S. auricularis—deer, dogs; S. caprae—goats; S. cohnii—chickens; S. devriesei—cattle; S. equorum—horses; S. felis—cats; S. fleurettii—goats; S. gallinarum—chickens, goats, pheasants; S. hyicus—pigs; S. lentus—goats, rabbits, sheep; S. lugdunensis—goats; S. nepalensis—goats; S. pasteuri—goats; S. pseudintermedius—dogs; S. rostri—pigs; and S. sciuri—dogs, goats.

Preferably, the Staphylococcus species is Staphylococcus aureus. This species is often referred to as “Golden Staph” and is a major cause of community and nosocomial infections in humans.

The Staphylococcus bacteria to be suppressed, inhibited, prevented, alleviated or treated; controlled or manipulated; prevented or delayed from developing antibiotic resistance; or the bacterial contamination prevented, removed or controlled may be derived from a single strain of a Staphylococcus species. Alternatively, the bacteria to be suppressed, inhibited, prevented, alleviated or treated; controlled or manipulated; prevented or delayed from developing antibiotic resistance; or the bacterial contamination prevented, removed or controlled may be a mixture of two or more strains of the same species of Staphylococcus.

Preferably, at least one of the Staphylococcus strains is an antibiotic resistant Staphylococcus strain. It may be a strain resistant to methicillin and/or vancomycin. By resistant, it is meant that the bacteria are less treatable with one or more antibiotics previously used to treat or prevent infection by those bacteria.

Preferably, the drug resistant Staphylococcus strain is resistant to more than one antibiotic, that is it is a multiply drug resistant Staphylococcus strain.

Preferably, the Staphylococcus species is S. aureus that is resistant to methicillin and/or vancomycin. Most preferably, the Staphylococcus species is methicillin resistant S. aureus (MRSA). MRSA is any strain of S. aureus that has developed, through the process of natural selection, and/or acquired resistance to β-lactam antibiotics, which include the penicillins (methicillin, dicloxacillin, nafcillin, oxacillin, etc.) and the cephalosporins, although they may be susceptible to the newest class of MRSA-active cephalosporins (e.g, ceftaroline). Strains of MRSA causing healthcare-associated infections are often multiply resistant to other commonly used antibacterial agents, including erythromycin, clindamycin, fluoroquinolones and tetracycline, while strains causing community-associated infections are often resistant only to β-lactam agents and erythromycin, and may be resistant to fluoroquinolones. MRSA strains with decreased susceptibility to vancomycin (minimum inhibitory concentration [MIC] 4-8 μg/ml) and strains fully resistant to vancomycin (MIC≥32 μg/ml) have been reported.

For example, the S. aureus may be chosen from the strains of the following list: ATCC BAA-1707, ATCC BAA-1717, ATCC BAA-1747, ATCC BAA-1754, ATCC BAA-1720, ATCC BAA-1761, ATCC BAA-1763, ATCC BAA-1764, ATCC BAA-1766, ATCC BAA-1768, ATCC 33591, ATCC 33592, ATCC 33591, ATCC 33592, ATCC R136, ATCC 700699, ATCC 10390, ATCC 13709, ATCC 27660, ATCC 29213, ATCC 33594, ATCC 49230, ATCC 6538P, ATCC 19636.

More preferably, the S. aureus may be chosen from the strains of the following list: ATCC BAA-1707, ATCC BAA-1717, ATCC BAA-1747, ATCC BAA-1754, ATCC BAA-1720, ATCC BAA-1761, ATCC BAA-1763, ATCC BAA-1764, ATCC BAA-1766, ATCC BAA-1768, ATCC 33591, ATCC 33592, ATCC 33591, ATCC 33592, ATCC R136, ATCC 700699. These S. aureus strains are classed as MRSAs.

The invention further provides the use of (i) at least one aryl antibiotic of Group I or pharmaceutically acceptable salts thereof for the manufacture of a medicament for the treatment of a bacterial infection, in combination with (ii) at least one antibiotic selected from the list comprising a penicillin or a derivative thereof, a cepham or a derivative thereof, vancomycin, linezolid, daptomycin and mupirocin. Preferably the antibiotic of (ii) is a penicillin or a derivative thereof or a cepham or a derivative thereof.

The invention provides the use of:
at least one aryl antibiotic of Group I or pharmaceutically acceptable salts thereof for the manufacture of a medicament for the treatment of a bacterial infection, in combination with a penicillin or a derivative thereof;
at least one aryl antibiotic of Group I or pharmaceutically acceptable salts thereof for the manufacture of a medicament for the treatment of a bacterial infection, in combination with a cepham or a derivative thereof;
at least one aryl antibiotic of Group I or pharmaceutically acceptable salts thereof for the manufacture of a medicament for the treatment of a bacterial infection, in combination with vancomycin;
at least one aryl antibiotic of Group I or pharmaceutically acceptable salts thereof for the manufacture of a medicament for the treatment of a bacterial infection, in combination with linezolid;
at least one aryl antibiotic of Group I or pharmaceutically acceptable salts thereof for the manufacture of a medicament for the treatment of a bacterial infection, in combination with daptomycin; and/or
at least one aryl antibiotic of Group I or pharmaceutically acceptable salts thereof for the manufacture of a medicament for the treatment of a bacterial infection, in combination with mupirocin.

The invention further provides the use of:
at least one aryl antibiotic of Formula A or Formula B or pharmaceutically acceptable salts thereof for the manufacture of a medicament for the treatment of a bacterial infection, in combination with at least one antibiotic selected from the list comprising penicillin or a derivative thereof or cepham or a derivative thereof;
at least one aryl antibiotic of Formula A or Formula B or pharmaceutically acceptable salts thereof for the manufacture of a medicament for the treatment of a bacterial infection, in combination with a penicillin or a derivative thereof;
at least one aryl antibiotic of Formula A or Formula B or pharmaceutically acceptable salts thereof for the manufacture of a medicament for the treatment of a bacterial infection, in combination with a cepham or a derivative thereof;
at least one aryl antibiotic of Formula A or Formula B or pharmaceutically acceptable salts thereof for the manufacture of a medicament for the treatment of a bacterial infection, in combination with at least one antibiotic chosen from the list comprising vancomycin, linezolid, daptomycin and mupirocin;
at least one aryl antibiotic of Formula A or pharmaceutically acceptable salts thereof for the manufacture of a medicament for the treatment of a bacterial infection, in combination with at least one antibiotic selected from the list comprising penicillin or a derivative thereof or cepham or a derivative thereof;
at least one aryl antibiotic of Formula A or pharmaceutically acceptable salts thereof for the manufacture of a medicament for the treatment of a bacterial infection, in combination with at least one antibiotic selected from the list comprising vancomycin, linezolid, daptomycin and mupirocin;
at least one aryl antibiotic of Formula B or pharmaceutically acceptable salts thereof for the manufacture of a medicament for the treatment of a bacterial infection, in combination with at least one antibiotic selected from the list comprising penicillin or a derivative thereof or cepham or a derivative thereof;
at least one aryl antibiotic of Formula A or pharmaceutically acceptable salts thereof for the manufacture of a medicament for the treatment of a bacterial infection, in combination with at least one antibiotic selected from the list comprising vancomycin, linezolid, daptomycin and mupirocin;
at least one aryl antibiotic of Formula A or pharmaceutically acceptable salts thereof for the manufacture of a medicament for the treatment of a bacterial infection, in combination with a penicillin or a derivative thereof;
at least one aryl antibiotic of Formula A or pharmaceutically acceptable salts thereof for the manufacture of a medicament for the treatment of a bacterial infection, in combination with a cepham or a derivative thereof;
at least one aryl antibiotic of Formula B or pharmaceutically acceptable salts thereof for the manufacture of a medicament for the treatment of a bacterial infection, in combination with penicillin or a derivative thereof or cepham or a derivative thereof;
at least one aryl antibiotic of Formula B or pharmaceutically acceptable salts thereof for the manufacture of a medicament for the treatment of a bacterial infection, in combination with a cepham or a derivative thereof.
at least one aryl antibiotic of Formula B or pharmaceutically acceptable salts thereof for the manufacture of a medicament for the treatment of a bacterial infection, in combination with vancomycin;
at least one aryl antibiotic of Formula B or pharmaceutically acceptable salts thereof for the manufacture of a medicament for the treatment of a bacterial infection, in combination with linezolid;
at least one aryl antibiotic of Formula B or pharmaceutically acceptable salts thereof for the manufacture of a medicament for the treatment of a bacterial infection, in combination with daptomycin; and/or
at least one aryl antibiotic of Formula B or pharmaceutically acceptable salts thereof for the manufacture of a medicament for the treatment of a bacterial infection, in combination with mupirocin.
   Preferably the bacterial infection is a Gram-positive bacterial infection, more preferably a bacterial infection caused by a Staphylococcus species.

The Staphylococcus species may be chosen from the list comprising: S. arlettae, S. agnetis, S. aureus, S. auricularis, S. capitis, S. caprae, S. carnosus, S. caseolyticus, S. chromogenes, S. cohnii, S. condimenti, S. croceolyticus, S. delphini, S. devriesei, S. epidermidis, S. equorum, S. faecalis, S. felis, S. fleurettii, S. gallinarum, S. haemolyticus, S. hominis, S. hyicus, S. intermedius, S. kloosii, S. leei, S. lentus, S. lugdunensis, S. lutrae, S. lyticans, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. roseus, S. saccharolyticus, S. saprophyticus, S. schleiferi, S. sciuri, S. simiae, S. simulans, S. stepanovicii, S. succinus, S. vitulinus, S. warneri, and S. xylosus.

More preferably, the Staphylococcus species is a species that can infect humans. For example, the specifies may be chosen from the list comprising: S. aureus, S. auricularis, S.carnosus, S. capitis, S. caprae, S. cohnii, S. epidermidis, S. haemolyticus, S.hyicus, S. leei, S. lugdunensis, S. pasteuri, S. pettenkoferi, S.saprophyticus, S. schleiferi, S. sciuri, S. simulans, S. warneri, and S. xylosus. More preferably, the species may be chosen from the list comprising: S.aureus, S.auricularis, S.carnosus, S.epidermidis, S.haemolyticus, S.hyicus, S.lugdunensis, S.saprophyticus, S.sciuri, S.simulans and S.warneri.

Alternatively, the Staphylococcus species may be a species that can infect an animal of economic, agricultural or social importance. For example, the Staphylococcus species may be chosen from the list comprising: S. arlattae; S. aureus; S. auricularis; S. caprae; S. cohnii; S. devriesei; S. equorum; S. felis; S. fleurettii; S. gallinarum; S. hyicus; S. lentus; S. lugdunensis; S. nepalensis; S. pasteuri; S. pseudintermedius; S. rostri; and S. sciuri.

The Staphylococcus may be derived from a single strain of a Staphylococcus species. Preferably, the Staphylococcus strain is a drug resistant Staphylococcus strain. Alternatively, the Staphylococcus may be a mixture of two or more strains of the same species of Staphylococcus. At least one of the strains may be resistant to methicillin and/or vancomycin. Preferably, the drug resistant Staphylococcus strain is resistant to more than one antibiotic, that is it is a multiply drug resistant Staphylococcus strain.

Preferably, the Staphylococcus species is Staphylococcus aureus. Preferably, the S. aureus is resistant to methicillin and/or vancomycin. Most preferably, the S. aureus is methicillin resistant S. aureus (MRSA).

The aryl antibiotic of Group I or the pharmaceutically acceptable salts thereof can be administered according to the invention before, simultaneously with or after the administration of the penicillin or a derivative thereof, cepham or a derivative thereof, vancomycin, linezolid, daptomycin or mupirocin. Substantially simultaneous or an exactly simultaneous administration of the combination partners is generally preferred.

The aryl antibiotic of Group I or pharmaceutically acceptable salts thereof and the penicillin or a derivative thereof, cepham or a derivative thereof, vancomycin, linezolid, daptomycin or mupirocin can be administered by any route of administration, preferably in the form of a pharmaceutical composition adapted to such a route. Dosage and route of administration should be determined by susceptibility of the causative organisms, severity and site of infection, and the specific condition of the patient and be selected accordingly. Preferred types of pharmaceutical compositions are, for example, oral, parenteral, enteral, intravenous, suppository, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive. The preferred route of administration is oral or transdermal.

The pharmaceutical composition may be administered as a solid dosage form such as a tablet, wafer, film, capsule, pill, granule, pellet, powder, and the like. The solid dosage form of the present invention may comprise a coating that is resistant to oral and/or gastric juices and dissolves as a function of the pH value of the release environment.

The pharmaceutical composition may also be administered as a liquid dosage form such as solutions, suspensions, dispersions, emulsions, foams, gels, oils, and the like.

The pharmaceutical composition of this invention may be administered by intravenous, intraarterial, or intramuscular injection of a liquid preparation. Suitable liquid compositions include solutions, suspensions, dispersions, emulsions, oils and the like.

The pharmaceutical composition may be administered topically to body surfaces, and is thus formulated in a form suitable for topical administration. Suitable topical compositions include liposomal beads, gels, ointments, creams, lotions, drops and the like. For topical administration, the anti-androgen agent and the antibiotic/anti-inflammatory agent are prepared and applied as solutions, suspensions, or emulsions in a physiologically acceptable diluent with or without a pharmaceutical carrier. For topical application, admixture of the compounds with conventional creams, lotions, or delayed release patches is acceptable. Such a cream or lotion may comprise any agent described herein, and, may be used to treat a dermatological disorder.

Preferably, the present invention provides a pharmaceutical composition comprising a combination of: (i) at least one aryl antibiotic of Group I or pharmaceutically acceptable salts thereof; and (ii) at least one antibiotic selected from the list comprising a penicillin or a derivative thereof, a cepham or a derivative thereof, vancomycin, linezolid, daptomycin and mupirocin and one or more excipients. Preferably the antibiotic of (ii) is a penicillin or a derivative thereof or a cepham or a derivative thereof.

This enhanced antibacterial activity allows the composition to have potent efficacy against a wide range of bacteria at levels where the antibiotics used individually may not be as effective.

Preferably, the present invention provides:
a pharmaceutical composition comprising a combination of: (i) at least one aryl antibiotic of Group I or pharmaceutically acceptable salts thereof; and (ii) a penicillin or a derivative thereof and one or more excipients;
a pharmaceutical composition comprising a combination of: (i) at least one aryl antibiotic of Group I or pharmaceutically acceptable salts thereof; and (ii) ar cepham or a derivative thereof and one or more excipients;
a pharmaceutical composition comprising a combination of: (i) at least one aryl antibiotic of Group I or pharmaceutically acceptable salts thereof; and (ii) vancomycin;
a pharmaceutical composition comprising a combination of: (i) at least one aryl antibiotic of Group I or pharmaceutically acceptable salts thereof; and (ii) linezolid;
a pharmaceutical composition comprising a combination of: (i) at least one aryl antibiotic of Group I or pharmaceutically acceptable salts thereof; and (ii) daptomycin; and/or
a pharmaceutical composition comprising a combination of: (i) at least one aryl antibiotic of Group I or pharmaceutically acceptable salts thereof; and (ii) mupirocin.

Preferably, the present invention provides:
a pharmaceutical composition comprising a combination of: (i) at least one aryl antibiotic of Formula A or Formula B or pharmaceutically acceptable salts thereof; and (ii) at least one antibiotic selected from the list comprising a penicillin or a derivative thereof, a cepham or a derivative thereof, vancomycin, linezolid, daptomycin and mupirocin and one or more excipients;
a pharmaceutical composition comprising a combination of: (i) at least one aryl antibiotic of Formula A or Formula B or pharmaceutically acceptable salts thereof; and (ii) a penicillin or a derivative thereof and one or more excipients;
a pharmaceutical composition comprising a combination of: (i) at least one aryl antibiotic of Formula A or Formula B or pharmaceutically acceptable salts thereof; and (ii) ar cepham or a derivative thereof and one or more excipients;
a pharmaceutical composition comprising a combination of: (i) at least one aryl antibiotic of Formula A or Formula B or pharmaceutically acceptable salts thereof; and (ii) vancomycin and one or more excipients;
a pharmaceutical composition comprising a combination of: (i) at least one aryl antibiotic of Formula A or Formula B or pharmaceutically acceptable salts thereof; and (ii) linezolid and one or more excipients;
a pharmaceutical composition comprising a combination of: (i) at least one aryl antibiotic of Formula A or Formula B or pharmaceutically acceptable salts thereof; and (ii) daptomycin and one or more excipients;
a pharmaceutical composition comprising a combination of: (i) at least one aryl antibiotic of Formula A or Formula B or pharmaceutically acceptable salts thereof; and (ii) mupirocin and one or more excipients;
a pharmaceutical composition comprising a combination of: (i) an aryl antibiotic of Formula A or pharmaceutically acceptable salts thereof; and (ii) at least one antibiotic selected from the list comprising penicillin or a derivative thereof or cepham or a derivative thereof and one or more excipients;
a pharmaceutical composition comprising a combination of: (i) an aryl antibiotic of Formula A or pharmaceutically acceptable salts thereof; and (ii) at least one antibiotic selected from the list comprising vancomycin, linezolid, daptomycin and mupirocin and one or more excipients;
a pharmaceutical composition comprising a combination of: (i) an aryl antibiotic of Formula B or pharmaceutically acceptable salts thereof; and (ii) at least one antibiotic selected from the list comprising penicillin or a derivative thereof or cepham or a derivative thereof and one or more excipients;
a pharmaceutical composition comprising a combination of: (i) an aryl antibiotic of Formula B or pharmaceutically acceptable salts thereof; and (ii) at least one antibiotic selected from the list comprising vancomycin, linezolid, daptomycin and mupirocin and one or more excipients;
a pharmaceutical composition comprising a combination of: (i) an aryl antibiotic of Formula A or pharmaceutically acceptable salts thereof; and (ii) at least one penicillin or a derivative thereof and one or more excipients;
a pharmaceutical composition comprising a combination of: (i) an aryl antibiotic of Formula A or pharmaceutically acceptable salts thereof; and (ii) at least one cepham or a derivative thereof and one or more excipients;
a pharmaceutical composition comprising a combination of: (i) an aryl antibiotic of Formula A or pharmaceutically acceptable salts thereof; and (ii) vancomycin and one or more excipients;
a pharmaceutical composition comprising a combination of: (i) an aryl antibiotic of Formula A or pharmaceutically acceptable salts thereof; and (ii) linezolid and one or more excipients;
a pharmaceutical composition comprising a combination of: (i) an aryl antibiotic of Formula A or pharmaceutically acceptable salts thereof; and (ii) daptomycin and one or more excipients;
a pharmaceutical composition comprising a combination of: (i) an aryl antibiotic of Formula A or pharmaceutically acceptable salts thereof; and (ii) mupirocin and one or more excipients;
a pharmaceutical composition comprising a combination of: (i) an aryl antibiotic of Formula B or pharmaceutically acceptable salts thereof; and (ii) at least one penicillin or a derivative thereof and one or more excipients;
a pharmaceutical composition comprising a combination of: (i) an aryl antibiotic of Formula B or pharmaceutically acceptable salts thereof; and (ii) at least one cepham or a derivative thereof and one or more excipients;
a pharmaceutical composition comprising a combination of: (i) an aryl antibiotic of Formula B or pharmaceutically acceptable salts thereof; and (ii) vancomycin and one or more excipients;
a pharmaceutical composition comprising a combination of: (i) an aryl antibiotic of Formula B or pharmaceutically acceptable salts thereof; and (ii) linezolid and one or more excipients;
a pharmaceutical composition comprising a combination of: (i) an aryl antibiotic of Formula B or pharmaceutically acceptable salts thereof; and (ii) daptomycin and one or more excipients;
a pharmaceutical composition comprising a combination of: (i) an aryl antibiotic of Formula B or pharmaceutically acceptable salts thereof; and (ii) mupirocin and one or more excipients;.

The preparation of pharmaceutical compositions that contain an active component is well understood in the art, for example by mixing, granulating, or tablet-forming processes. The active agent is often mixed with excipients that are pharmaceutically acceptable and compatible with the active agent.

For oral administration, the aryl antibiotics of Group I or pharmaceutically acceptable salts thereof and/or the antibiotics selected from the list comprising a penicillin or a derivative thereof, a cepham or a derivative thereof, vancomycin, linezolid, daptomycin and mupirocin of this invention are mixed with excipients and additives customary for this purpose, such as vehicles, stabilizers, or inert diluents, and converted by customary methods into suitable forms for administration, such as tablets, coated tablets, hard or soft gelatine capsules, aqueous, alcoholic or oily solutions.

For parenteral administration, the aryl antibiotics of Group I or pharmaceutically acceptable salts thereof and/or the antibiotics selected from the list comprising a penicillin or a derivative thereof, a cepham or a derivative thereof, vancomycin, linezolid, daptomycin and mupirocin are converted into a solution, suspension, or emulsion, if desired with the excipients and substances customary and suitable for this purpose, for example, solubilizers or other like agents.

A pharmaceutical product according to the invention can, for example, comprise one or more than one dosage unit of at least one aryl antibiotic of Group I or pharmaceutically acceptable salts thereof and separately one or more than one other dosage unit of a penicillin or a derivative thereof, a cepham or a derivative thereof, vancomycin, linezolid, daptomycin or mupirocin. By the way of example, a pharmaceutical product of the invention may comprise two separate packages, each of them comprising a pharmaceutical composition comprising just one of the combination partners in an appropriate dosage form.

Another embodiment of the pharmaceutical product according to the invention comprises one or more than one dosage unit, and each dosage unit comprises both at least one aryl antibiotic of Group I or pharmaceutically acceptable salts thereof and a penicillin or a derivative thereof, a cepham or a derivative thereof, vancomycin, linezolid, daptomycin or mupirocin. Thus the single pharmaceutical product contains both of the combination partners in an appropriate dosage form. Such a fixed dose combination generally comprises at least one aryl antibiotic of Group I or pharmaceutically acceptable salts thereof and the penicillin or a derivative thereof, cepham or a derivative thereof, vancomycin, linezolid, daptomycin or mupirocin as well as a pharmaceutically acceptable carrier and optionally appropriate further excipients as typical for the respective dosage form.

The pharmaceutical products according to the present invention comprise at least one aryl antibiotic of Group I or pharmaceutically acceptable salts thereof and the penicillin or a derivative thereof, cepham or a derivative thereof, vancomycin, linezolid, daptomycin or mupirocinin an appropriate weight ratio, e.g. in a weight ratio of 1024:1 to 1:1024, preferably from 256:1 to 1:256, more preferably from 64:1 to 1:64.

The dosage of at least one aryl antibiotic of Group I or pharmaceutically acceptable salts thereof and the penicillin or a derivative thereof, cepham or a derivative thereof, vancomycin, linezolid, daptomycin or mupirocin for said treatment can vary within wide limits and will be fitted in each particular case to the individual requirements of the patient to be treated and to the bacterial populations to be controlled. In general, a dosage of about 0.01 to about 4 g, e.g. about 0.5 to about 2 g, of total antibiotic administered one to four times over a 24 hours period should be appropriate.

The composition can contain from 0.1% to 99% by weight, preferably 10%-90% by weight, of each of the active ingredients. If the compositions contain dosage units, each unit preferably contains from 50 mg to 4 g of each active substance.

The composition may be administered one a day, twice a day, three times a day or more often. Alternatively, the composition may be administered weekly, monthly etc, particularly if the composition is administered in the form of a slow release dosage. Alternatively, if the composition is to be administered via parenteral administration, it may be administered continuously over a period of hours, days or weeks. The choice of dosage administration timing is reliant on factors such as the route of administration (e.g. oral, parenteral, topical, infusion etc), the release rate of the dosage (e.g. slow release, rapid release), the site of the infection, the nature of the bacteria being treated and/or the subject being administered the dosage. Each of these factors will be taken into consideration when designing a dosage regime.

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

Topical Compositions

The pharmaceutical composition may be adapted for topical application. In this regard, various topical delivery systems may be appropriate for administering the compositions of the present invention depending up on the preferred treatment regimen. Topical compositions may be produced by dissolving or combining the antibiotics of the present invention in an aqueous or non-aqueous carrier. In general, any liquid, cream, or gel or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc, which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the antibiotics are added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the antibiotics can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the antibiotics are added to the composition at the desired concentration at a point preceding addition of the gelling agent.

The amount of antibiotic compounds incorporated into a topical composition is not critical; the concentration should be within a range sufficient to permit ready application of the composition such that an effective amount of the antibiotic is delivered.

Oral Compositions

The pharmaceutical composition may be adapted for oral delivery. In this regard, the antibiotics can be administered as an oral preparation adapted in such a manner that facilitates delivery of a therapeutically effective concentration of the antibiotics.

The effective dosages of the antibiotics, when administered orally, must take into consideration the diluent, preferably water. The composition preferably contains 0.05% to about 100% by weight active ingredient and more preferably about 10% to about 80% by weight. When the compositions are ingested, desirably they are taken on an empty stomach.

Contemplated for use herein are oral solid dosage forms including tablets, capsules, pills, films, wafers, troches or lozenges, cachets or pellets. Also, liposomal or proteinoid encapsulation may be used to formulate the present compositions. Liposomal encapsulation may be used and the liposomes may be derivatised with various polymers. In general, the composition will include the antibiotic compounds and inert ingredients that allow for protection against the stomach environment and release of the biologically active material in the intestine.

The location of release may be the stomach, the small intestine (the duodenum, the jejunem, or the ileum), or the large intestine. One skilled in the art has available compositions that will not dissolve in the stomach, yet will release the material in the duodenum or elsewhere in the intestine. Preferably, the release will avoid the deleterious effects of the stomach environment, either by protection of the antibiotics or by release of the antibiotics beyond the stomach environment, such as in the intestine.

To ensure full gastric resistance, a coating impermeable to at least pH 5.0 may be used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S and Shellac. These coatings may be used as mixed films.

A coating or mixture of coatings that are not intended for protection against the stomach can also be used on tablets. This can include sugar coatings, or coatings that make the solid dosage form easier to swallow. Capsules may consist of a hard shell (such as gelatine) for delivery of dry therapeutic i.e. powder; for liquid forms, a soft gelatine shell may be used. The shell material of cachets could be thick starch or other edible paper. For pills, lozenges, moulded tablets or tablet triturates, moist massing techniques can be used.

One may dilute or increase the volume of the composition with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the composition of the antibiotic compounds into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite may all be used. Another form of the disintegrants is insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the composition together to form a hard tablet and include materials from natural products such as acacia, tragacanth, starch and gelatine. Others include methylcellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the compound.

An antifrictional agent may be included in the composition to prevent sticking during the composition process. Lubricants may be used as a layer between the compound and the die wall and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights and Carbowax 4000 and 6000.

Glidants that might improve the flow properties of the composition during composition and to aid rearrangement during compression might be added. The glidants may include starch, talc, pyrogenic silica and hydrated silicoaluminate.

To aid dissolution of the compound, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the composition either alone or as a mixture in different ratios.

Controlled release compositions may be desirable. Controlled release has several distinct variants such as sustained release where prolonged release is intended, pulse release, delayed release (e.g. to target different regions of the GI tract) etc. A distinction of controlled release is that not only prolongs action but it attempts to maintain drug levels within the therapeutic window to avoid potentially hazardous peaks in drug concentration following ingestion or injection and to maximize therapeutic efficiency. The compounds can be incorporated into an inert matrix that permits release by either diffusion or leaching mechanisms i.e., gums. Slowly degenerating matrices may also be incorporated into the composition. Another form of a controlled release composition is by a method based on the Oros therapeutic system (Alza Corp.), i.e. the composition is enclosed in a semipermeable membrane which allows water to enter and push the composition out through a single small opening due to osmotic effects. Some enteric coatings also have a delayed release effect.

The pharmaceutical composition of the present invention may be formulated for sustained release. Sustained release is defined herein to mean that the aryl antibiotics of Group I or pharmaceutically acceptable salts thereof and/or the antibiotics selected from the list comprising penicillin or a derivative thereof, cepham or a derivative thereof, vancomycin, linezolid, daptomycin or mupirocinbecome available for bio-absorption in the gastrointestinal tract over a prolonged period of time. The release rate of the active agents is primarily controlled by dissolution of the active agents in gastrointestinal fluid and subsequent diffusion out of the tablet or capsule independent of pH, but can also be influenced by physical processes of disintegration and erosion of the tablet or capsule. Pharmaceutical compositions according to the invention achieve a therapeutic blood/plasma concentration of the aryl antibiotics of Group I or pharmaceutically acceptable salts thereof and/or the antibiotics selected from the list comprising penicillin or a derivative thereof, cepham or a derivative thereof, vancomycin, linezolid, daptomycin or mupirocin in an individual for at least about 8 to about 14 hours from a single dose. The aryl antibiotics of Group I or pharmaceutically acceptable salts thereof and/or the antibiotics selected from the list comprising penicillin or a derivative thereof, cepham or a derivative thereof, vancomycin, linezolid, daptomycin or mupirocin may be released from the tablet or capsule to result in a therapeutic blood/plasma concentration for at about 8, 9, 10, 11, 12, 13 or 14 hours from a single dose.

The pharmaceutical composition may be an immediate release composition, i.e., a composition in which the whole quantity of the aryl antibiotics of Group I or pharmaceutically acceptable salts thereof and/or the antibiotics selected from the list comprising penicillin or a derivative thereof, cepham or a derivative thereof, vancomycin, linezolid, daptomycin or mupirocin is released immediately after administration. Such immediate release compositions disintegrate readily to form a suspension or solution of the active agents after mixing with the saliva, which is easily swallowed by the patients. These are particularly suitable for children or aged patients who have difficulty in chewing and/or swallowing an intact tablet/capsule.

The aryl antibiotics of Group I or pharmaceutically acceptable salts thereof and/or the antibiotics selected from the list comprising penicillin or a derivative thereof, cepham or a derivative thereof, vancomycin, linezolid, daptomycin or mupirocin may be formulated as micronized or non-micronized particles. The non-micronized particles refer to particles having a particle size between 20-90 microns. The micronized particles refer to particles having a particle size between 1-20 microns. The particles may be formulated as solid or liquid dosage forms for oral administration.

A mix of materials might be used to provide the optimum film coating. Film coating may be carried out in a pan coater or in a fluidised bed or by compression coating.

The antibiotic compounds can be included in the composition as fine multi-particulates in the form of granules or pellets of particle size about 1 mm. The composition of the material for capsule administration could also be as a powder, lightly compressed plugs or even as tablets. The compound could be prepared by compression.

Injectable Compositions

The antibiotic compounds can also be formulated for parenteral delivery. Pharmaceutical forms suitable for injectable use include: sterile aqueous solutions (where water-soluble) or dispersions and sterile powders for the extemporaneous preparation of sterile injectable solutions or dispersion including nanosuspensions and nanocrystals. These solutions or suspensions can be prepared from sterile powders, granules or lyophilizates. Pharmaceutical compositions for parenteral administration can also include concentrates or solutions for further dilutions (e.g. for infusions). Alternatively, the antibiotic compounds of the invention may be encapsulated in liposomes and delivered in injectable solutions to assist their transport across cell membrane. The solution may be a solvent or dispersion medium containing, for example, water, ethanol, polyol (for example, glycerol, propylene glycol and liquid polyethylene glycol and the like), suitable mixtures thereof and vegetable oils. The antibiotic compounds can be dissolved in sterile water or in various sterile buffers that may contain, but are not limited to contain, sodium chloride, polyethylene glycol, propylene glycol, ethanol, sucrose, glucose, arginine, lysine, citric acid, lactic acid phosphoric acid and corresponding salts. Proper fluidity may be maintained, for example, by the use of a coating such as lecithin, by the maintenance of the required particle size in the case of dispersion and by the use of surfactants. Prolonged absorption of the injectable compositions containing antibiotics can be brought about by the use in the compositions of agents delaying absorption, for example, aluminium monostearate and gelatine.

Sterile injectable solutions may be prepared by incorporating the antibiotic compounds in the required amount in an appropriate solvent with various of the other ingredients enumerated above, as required, followed by filtered sterilisation. Generally, dispersions are prepared by incorporating the compound into a sterile vehicle that contains the basic dispersion medium and the other ingredients. In the case of sterile powders for the preparation of sterile injectable solutions, the preferred methods of preparation are vacuum drying and freeze-drying techniques that yield a powder of the compound plus any additional desired ingredient from previously sterile-filtered solution thereof.

Thus, the present invention also provides an injectable, stable, sterile composition comprising the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin, in one or more unit dosage forms in a sealed container. The composition may be provided in lyophilised form capable of being reconstituted with a suitable pharmaceutically acceptable carrier to form a liquid composition suitable for injection thereof into a subject. The unit dosage form typically comprises from about 10 mg to about 10 grams of the antibiotic or antibiotics. When the antibiotic or antibioticsare substantially water-insoluble, a sufficient amount of emulsifying agent which is physiologically acceptable may be employed in sufficient quantity to emulsify the antibiotic or antibiotics in an aqueous carrier. One such useful emulsifying agent is phosphatidyl choline.

Aerosols

Pharmaceutical compositions are also provided which are suitable for administration as an aerosol, by inhalation. These compositions comprise a solution or suspension of the antibiotic or antibioticsor a plurality of solid particles of the antibiotic or antibiotics. The antibiotic or antibiotics are the (i) aryl antibiotics of Group I or pharmaceutically acceptable salts thereof and (ii) the antibiotic selected from the list comprising a penicillin or a derivative thereof, a cepham or a derivative thereof, vancomycin, linezolid, daptomycin and mupirocin. The desired composition may be placed in a small chamber and nebulized. Nebulization may be accomplished by compressed air or by ultrasonic energy to form a plurality of liquid droplets or solid particles comprising the antibiotic or antibiotics.

The solid particles can be obtained by processing solid antibiotic or antibiotics, in any appropriate manner known in the art, such as by micronization. Commercial nebulizers are also available to provide liquid droplets of any desired size.

The liquid droplets or solid particles should have a particle size in the range of about 0.5 to about 5 microns, preferably from about 1 to about 2 microns. Most preferably, the size of the solid particles or droplets will be from about 1 to about 2 microns. Such particles or droplets may be dispensed by commercially available nebulisers or by other means known to the skilled person.

When the pharmaceutical composition suitable for administration as an aerosol is in the form of a liquid, the composition will comprise a water-soluble form of the antibiotic or antibiotics, in a carrier that comprises water. A surfactant may be present which lowers the surface tension of the composition sufficiently to result in the formation of droplets within the desired size range when subjected to nebulization.

In addition, the pharmaceutical composition may also include other agents. For example, preservatives, co-solvents, surfactants, oils, humectants, emollients, chelating agents, dyestuffs, stabilizers or antioxidants may be employed. Water soluble preservatives that may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. The surfactant may preferably be polysorbate 80. Other suitable additives include lubricants and slip agents, such as, for example, magnesium stearate, stearic acid, talc and bentonites, substances which promote disintegration, such as starch or cross linked polyvinylpyrrolidone, binders, such as, for example, starch, gelatin or linear polyvinylpyrrolidone, and dry binders, such as microcrystalline cellulose.

Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylatedhydroxyanisole, butylated hydroxytoluene, etc. The indications, effective doses, compositions, contraindications, vendors etc, of the antibiotic compounds in the compositions are available or are known to one skilled in the art. These antibiotic compounds may be present in individual amounts of from about 0.001% to about 5% by weight and preferably about 0.01% to about 2%.

Electrolytes such as, but not limited to, sodium chloride and potassium chloride may also be included in the composition.

Further, the compositions may contain microbial preservatives. Useful microbial preservatives include methylparaben, propylparaben, benzyl alcohol, phenoxyethanol and hydroxyacetophenone. The microbial preservative is typically employed when the composition is placed in a vial designed for multidose use.

Excipients which may be used are all the physiologically acceptable solid inert substances, either inorganic or organic in nature. Inorganic substances are, for example, sodium chloride, carbonates, such as calcium carbonate, bicarbonates, aluminium oxides, silicic acids, aluminas, precipitated or colloidal silicon dioxide and phosphates. Organic substances are, for example, sugars, cellulose, foodstuffs and feedstuffs, such as milk powder, animal flours, cereal flours and shredded cereals and starches.

Finally, it will be appreciated that the compositions of the present invention may comprise a plurality of antibiotic compounds as described herein.

The pharmaceutical composition may be formulated with, but not limited to, pharmaceutically acceptable carriers or diluents, fillers, polymers, glidants, and lubricants.

Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and polyvinyl pyrrolidone. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy ((Lachman et al., eds., 3.sup.rd edition, Lippincott Williams & Wilkins, 1986); Encyclopedia of Pharmaceutical Technology (Swarbrick and Boylan, eds., 2nd edition, Marcel Dekker, 2002).

The fillers can be chosen from, but are not limited to, powdered cellulose, sorbitol, mannitol, various types of lactose, phosphates and the like.

The polymers can be chosen from, but not limited to, hydrophilic or hydrophobic polymers such as derivatives of cellulose (for example methylcellulose, hydroxypropyl cellulose, hypromellose, ethylcellulose); polyvinylpirolidone (for example povidone, crospovidone, copovidone); polymethacrylates (for example Eudragit RS, RL); lypophillic components (for example glyceryl monostearate, glyceryl behenate); and various other substances such as for example hydroxypropyl starch, polyethylene oxide, carrageenan and the like. Most commonly, hydrophilic swelling polymers of suitable viscosity such as hypromellose are used, preferably in amounts above 5%, and more preferably above 8%.

Glidants can be chosen from, but not limited to, colloidal silicon dioxide, talc, magnesium stearate, calcium stearate, aluminium stearate, palmitic acid, stearic acid, stearol, cetanol, polyethylene glycol and the like.

Lubricants can be chosen from, but not limited to, stearic acid, magnesium stearate, calcium stearate, aluminium stearate, sodium stearyl fumarate, talc, hydrogenated castor oil, polyethylene glycols and the like.

One of ordinary skill in the art will appreciate that the individual components of the present invention may change depending on the physical and chemical qualities needed for the pharmaceutical compositions in a given process and/or application to which the pharmaceutical compositions will be applied.

### General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

### Materials and Equipment

### 1. Test Substances and Dilutions

Test substances, Ramizol and Chlopam, in powder form were provided by Boulos & Cooper Pharmaceuticals Pty Ltd. Test substances from Boulos & Cooper Pharmaceuticals were all dissolved and diluted with 100% DMSO in 2-fold dilutions. The commercial antibiotics were dissolved and diluted in the CLSI specified diluent.

TABLE 1

| | | Solubility | | Light | Temperature | Formulation |
|---|---|---|---|---|---|---|
| Test Compound | Vehicle | (a) | Color | Protection (b) | (c) | mg/mL |
| Ramizol | DMSO | S | Pale Yellow | N | RT | 6.4 and dilutions |
| Chlopam | DMSO | S | Colorless | N | RT | 6.4 and dilutions |
| Cefepime | PBS | S | Colorless | N | 4° C. | 25.6 and dilutions |
| Daptomycin | WFI | S | Colorless | N | 4° C. | 0.05 and dilutions |
| Linezolid | WFI | I | White | Y | RT | 0.8 and dilutions |
| Mupirocin | WFI | S | Colorless | N | RT | 0.1 and dilutions |
| Oxacillin | WFI | S | Colorless | N | RT | 25.6 and dilutions |
| Vancomycin | WFI | S | Colorless | N | RT | 3.2 and dilutions |

### 2. Medium and Chemicals

Bacto agar (Cat#214040, BD DIFCO, USA), Cation-adjusted Mueller Hinton Broth II (Cat#212322, BD DIFCO, USA), Cefepime hydrochloride (Cat#A3737, Sigma, USA), Daptomycin (Cat#35710, Chempacific, USA), Dimethyl sulfoxide (Cat#1.02931.1000, Merck, Germany), Linezolid (ZYVOX®, Pfizer, USA), Mupirocin (Cat#M7694, Sigma, USA), Oxacillin (Lot#26P720, chemical & Pharmaceutical, China), Vancomycin (Cat#V2002, Sigma, USA) and Water for injection (WFI) (Tai-Yu, Taiwan).

### 3. Equipment and Plastic-Ware

96-well round bottom polystyrene plate (NUNC, USA), Absorbance microplate readers (Tecan, Infinite F50, USA), Biological safety cabinet (NuAire, USA), Incubator (Firstek Scientific, Taiwan), Petri dish (Gelman, USA), Pipetman (Rainin, USA), and Ultra-Low temperature freezer (NuAire, USA).

### Methods

### Inoculum Preparation

Staphylococcus aureus (ATCC 29213) and Staphylococcus aureus (BAA-1717), grown on solid agar medium were indicated in the following table. Colonies were suspended in PBS. The absorbance of each suspension was measured with a spectrophotometer, and then the suspension was adjusted to 1×10⁶ CFU/mL. The testing inoculum was prepared within 20 minutes before adding to wells of the test plate. Final cellular density in the checkerboard assays was 5×10⁵ CFU/mL.

TABLE 2

| | | | | Isolates growth/Assay | | | |
|---|---|---|---|---|---|---|---|
| | | | | plates condition | | | |
| Organism | Group | Strain ID | Characteristics | Temp. | | | |
| Culture Medium | | Time (hr) | | (° C.) | | Assay Medium | |
| S. aureus | G(+) | ATCC 29213 | CLSI | NA | 20-24 | 35 ± 2 | CAMHB |
| | | | control, | | | | |
| | | | methicillin | | | | |
| | | | susceptible | | | | |
| S. aureus | G(+) | ATCC BAA- | USA 300, | NA | 20-24 | 35 ± 2 | CAMHB |
| | | 1717 | MRSA | | | | |

a. Antibiotic resistance mechanisms are based on information from the strain source
b. Medium: CAMHB: Cation-adjusted Mueller Hinton Broth II;
NA: Nutrient Agar

### Checkerboard Assays

The combination effect of antibacterial substances administered together was evaluated by an in vitro checkerboard combination method with Staphylococcus aureus (ATCC 29213) and Staphylococcus aureus (BAA-1717). Ramizol and Chlopam were tested alone in 11 points by two-fold serial titration and in combination with different commercial antibiotics which were tested in a 7-point titration alone and in combination with Ramizol and Chlopam using the checkerboard technique as recommended by the Clinical & Laboratory Standards Institute (CLSI. Methods for Dilution In Antimicrobial Susceptibility Tests for Bacteria That Grow Aerobically; Approved Standard-Eighth Edition. CLSI document M07-A8 (ISBN 1-56238-689-1). Vol. 29 No. 2. Clinical and Laboratory Standards Institute, USA, 2009). The commercial antibiotics were also tested alone. Twenty-four checkerboard tests were performed.

The starting concentration of Ramizol, Chlopam, and the commercial antibiotics was four fold of the MIC. The testing range for each combination was described in the following summary tables.

Compound dilutions of Ramizol and Chlopam were performed in 100% DMSO and the final DMSO concentration in all test wells was 2%. Growth control wells also included 2% DMSO. CLSI protocol was followed for medium, inoculum preparation, and endpoint reading. The commercial antibiotics were dissolved and diluted in the CLSI specified diluent. The final inoculum count for each strain was 5×10⁵ CFU/mL.

Checkerboard Group 1: Test with Staphylococcus aureus (ATCC 29213)

Chlopam and Ramizol were tested in an eleven-point titration (started from 4× MIC) alone and in combination with Cefepime, Daptomycin, Linezolid, Mupirocin, Oxacillin and Vancomycin (started from 4× MIC, 7 point titration). Cefepime, Daptomycin, Linezolid, Mupirocin, Oxacillin and Vancomycin were also tested alone. Each test substance was dissolved and diluted in 100% DMSO or specified solvent. Each test substance dilution (2 μL) was added to 96 μL broth CAMHB. CAMHB seeded with S. aureus ATCC 29213 at 1×10⁶ CFU/mL (100 μL) was then added to the plate to generate a final cell count at 5×10⁵ CFU/mL.

Checkerboard Group 2: Test with Staphylococcus aureus (BAA-1717)

Chlopam and Ramizol were tested in an eleven-point titration (started from 4× MIC) alone and in combination with Cefepime, Oxacillin, Vancomycin, Linezolid, Daptomycin and Mupirocin (started from 4× MIC, 7 point titration). Cefepime, Oxacillin, Vancomycin, Linezolid, Daptomycin and Mupirocin were also tested alone. Each test substance was dissolved and diluted in 100% DMSO or specified solvent. Each test substance dilution (2 μL) was added to 96 μL broth CAMHB. CAMHB seeded with S. aureus BAA-1717 at 1×10⁶ CFU/mL (100 μL) was then added to the plate to generate a final cell count at 5×10⁵ CFU/mL.

Assay plates were incubated and MIC endpoints were read as described in the MIC assay. FIC (Fraction Inhibitory Concentration) and FIC Index (FICI) values for each test condition and the average of FICI values of all pairwise combinations were calculated to determine if synergy or antagonism existed between the two substances. The FIC index was determined by calculating the sum of the ratios of MICs for both substances. Arithmetically, the FIC Index of a combination of Substance 1 and Substance 2 was defined as following: FICI=Σ[FIC(Substance 1)+FIC(Substance 2)]=[(MIC of Substance 1 in combination/MIC of Substance 1 alone)+(MIC of Substance 2 in combination/MIC of Substance 2 alone)]. Synergy is defined as the FICI (Σ)≤0.5; additivity as FICI (Σ)>0.5 to ≤1; indifference as FICI (Σ)>1 to ≤4; antagonism is defined as the FICI (Σ)>4 (Zinner and Blaser, J. Journal of Antimicrobial Chemotherapy (1986), 17, 1-5; Amin, et al BMC Complementary & Alternative Medicine (2015), 15, 59; Zuo, et al. Molecules (2011), 16, 9819-9826).

TABLE 3
Concentration ranges of antibiotics (μg/mL)
used for the checkerboard method.

| | CHL | CEF | OXA | VAN |
|---|---|---|---|---|
| S. aureus | 4-0.0039 | 8-0.125 | 1-0.015625 | 4-0.0625 |
| ATCC 29213 | | | | |
| S. aureus | 2-0.00195 | 512-8 | 512-8 | 2-0.03125 |
| ATCC BAA-1717 | | | | |
| | RZL | LIN | DAP | MUP |
| S. aureus | 8-0.125 | 16-0.25 | 1-0.015625 | 2-0.03125 |
| ATCC 29213 | | | | |
| S. aureus | 8-0.125 | 8-0.125 | 1-0.015625 | 1-0.015625 |
| ATCC BAA-1717 | | | | |

Abbreviations:
CHL (Chlopam),
CEF (cefepime),
OXA (oxacillin),
VAN (vancomycin),
LIN (linezolid),
DAP (daptomycin),
MUP (Mupirocin),
RZL (ramizol),

### Results

### Chlopam

TABLE 4
Summary of MICs of antibiotics and mean FICIs (in brackets) of
antibiotic combinations with Chlopam against S. aureus.

| Species | Chlopam | Cefepime | Oxacillin | Vancomycin |
|---|---|---|---|---|
| S. aureus | 0.5 | 4 (1.1) | 0.5 (1.0) | 1 (1.0) |
| ATCC 29213 | | | | |
| S. aureus | 1 | 256 (0.8) | 128 (1.2) | 1 (1.2) |
| ATCC BAA-1717 | | | | |
| Species | Chlopam | Linezolid | Daptomycin | Mupirocin |
| S. aureus | 0.5 | 2 (0.9) | 0.5 (1.1) | 0.5 (1.1) |
| ATCC 29213 | | | | |
| S. aureus | 1 | 1 (1.2) | 0.5 (1.2) | 0.5 (1.2) |
| ATCC BAA-1717 | | | | |

TABLE 5
Summary of individual antibiotic combinations with Chlopam leading
to a synergistic effect. MICs of antibiotics and FICIs (in brackets).

| Species | Chlopam | Oxacillin | Cefepime | FICI |
|---|---|---|---|---|
| S. aureus ATCC 29213 | 0.125 | 0.125 | | 0.5 |
| S. aureus ATCC BAA-1717 | 0.25 | | 64 | 0.5 |
| | 0.25 | | 32 | 0.4 |
| | 0.25 | | 16 | 0.3 |
| | 0.50 | | 8 | 0.5 |

TABLE 6
Summary of individual antibiotic combinations with Chlopam leading to
an additive effect; MICs of antibiotics and FICIs (in brackets).

| Species | Chlopam | Cefepime) | Oxacillin | Vancomycin |
|---|---|---|---|---|
| S. aureus | 0.003906 | 4 (1.0) | 0.5 (1.0) | 1 (1.0) |
| ATCC 29213 | 0.0078125 | 4 (1.0) | 0.5 (1.0) | 1 (1.0) |
| | 0.015625 | 4 (1.0) | 0.5 (1.0) | 1 (1.0) |
| | 0.125 | | 0.25 (0.8) | 0.5 (0.8) |
| | 0.25 | 2 (1.0) | 0.125 (0.8) | 0.5 (1.0) |
| | 0.5 | 0.125 (1.0) | 0.01563 (1.0) | |
| S. aureus ATCC | 0.001953 | 256 (1.0) | 128 (1.0) | 1 (1.0) |
| BAA-1717 | 0.003906 | 256 (1.0) | 128 (1.0) | 1 (1.0) |
| | 0.0078125 | 256 (1.0) | 128 (1.0) | 1 (1.0) |
| | 0.015625 | 256 (1.0) | | 1 (1.0) |
| | 0.03125 | 256 (1.0) | | |
| | 0.125 | 128 (0.6) | | |
| | 0.5 | | | 0.031 (1.0) |
| Species | Chlopam | Linezolid | Daptomycin | Mupirocin |
| S. aureus | 0.003906 | 2 (1.0) | 0.5 (1.0) | 0.5 (1.0) |
| ATCC 29213 | 0.0078125 | 2 (1.0) | 0.5 (1.0) | 0.5 (1.0) |
| | 0.015625 | 2 (1.0) | 0.5 (1.0) | 0.5 (1.0) |
| | 0.03125 | | | |
| | 0.125 | 1 (0.8) | | |
| | 0.25 | 0.5 (0.8) | 0.25 (1.0) | 0.25 (1.0) |
| | 0.5 | 0.25 (0.6) | 0.016 (1.0) | |
| S. aureus | 0.001953 | 1 (1.0) | 0.5 (1.0) | 0.5 (1.0) |
| ATCC BAA-1717 | 0.003906 | 1 (1.0) | 0.5 (1.0) | 0.5 (1.0) |
| | 0.0078125 | 1 (1.0) | 0.5 (1.0) | 0.5 (1.0) |
| | 0.015625 | 1 (1.0) | 0.5 (1.0) | 0.5 (1.0) |
| | 0.5 | | 0.016 (1.0) | 0.016 (1.0) |

### Ramizol

TABLE 7
Summary of MICs of antibiotics and mean FICIs (in brackets) of
antibiotics combinations with Ramizol against S. aureus.

| Species | Ramizol | Cefepime | Oxacillin | Vancomycin |
|---|---|---|---|---|
| S. aureus | 4 | 4 (1.0) | 0.5 (1.2) | 1 (1.2) |
| ATCC 29213 | | | | |
| S. aureus ATCC | 4 | 256 (0.696) | 128 (0.9) | 1 (1.2) |
| BAA-1717 | | | | |
| Species | Ramizol | Linezolid | Daptomycin | Mupirocin |
| S. aureus ATCC | 4 | 4 (1.1) | 0.5 (1.0) | 0.5 (1.1) |
| 29213 | | | | |
| S. aureus ATCC | 4 | 1 (1.2) | 0.5 (1.1) | (1.2) |
| BAA-1717 | | | | |

TABLE 8
Summary of individual antibiotic combinations with Ramizol leading to a
synergistic effect. MICs of antibiotics and FICIs (in brackets).

| Species | Ramizol | Cefepime | FICI |
|---|---|---|---|
| S. aureus ATCC BAA-1717 | 0.125 | 128 | 0.5 |
| | 1 | 64 | 0.5 |
| | 2 | 8 | 0.5 |

TABLE 9
Summary of individual antibiotic combinations with Ramizol leading to an
additive effect. MICs of antibiotics and FICIs (in brackets).

| Species | Ramizol | Cefepime | Oxacillin | Vancomycin |
|---|---|---|---|---|
| S. aureus ATCC 29213 | 0.015625 | 4 (1.0) | 0.5 (1.0) | 1 (1.0) |
| | 0.03125 | 4 (1.0) | 0.5 (1.0) | 1 (1.0) |
| | 0.0625 | 4 (1.0) | 0.5 (1.0) | 1 (1.0) |
| | 0.125 | 4 (1.0) | 0.5 (1.0) | 1 (1.0) |
| | 1 | 2 (0.8) | | |
| | 2 | 1 (0.8) | | |
| | 4 | 0.125 (1.0) | 0.016 (1.0) | |
| S. aureus ATCC | 0.015625 | 256 (1.0) | 128 (1.0) | 1 (1.0) |
| BAA-1717 | 0.03125 | 256 (1.0) | 128 (1.0) | 1 (1.0) |
| | 0.0625 | 256 (1.0) | 128 (1.0) | 1 (1.0) |
| | 0.125 | | | 1 (1.0) |
| | 0.25 | 128 (0.6) | | |
| | 0.5 | 128 (0.6) | | |
| | 1 | | 64 (1.0) | |
| | 1 | | 32 (0.8) | |
| | 1 | | 16 (0.6) | |
| | 1 | | 8 (0.6) | |
| | 2 | 32 (0.6) | | |
| | 2 | 16 (0.6) | | |
| | 4 | | | 0.031 (1.0) |
| Species | Ramizol | Linezolid | Daptomycin | Mupirocin |
| S. aureus ATCC 29213 | 0.015625 | 4 (1.0) | 0.5 (1.0) | 0.5 (1.0) |
| | 0.03125 | 4 (1.0) | 0.5 (1.0) | 0.5 (1.0) |
| | 0.0625 | 4 (1.0) | 0.5 (1.0) | 0.5 (1.0) |
| | 0.125 | 4 (1.0) | 0.5 (1.0) | 0.5 (1.0) |
| | 0.25 | 4 (1.0) | | 0.5 (1.0) |
| | 2 | | 0.25 (1.0) | |
| | 2 | | 0.125 (0.8) | |
| | 2 | | 0.063 (0.6) | |
| | 2 | | 0.031 (0.6) | |
| | 4 | | 0.016 (1.0) | |
| S. aureus ATCC | 0.015625 | 1 (1.0) | 0.5 (1.0) | 0.5 (1.0) |
| BAA-1717 | 0.03125 | 1 (1.0) | 0.5 (1.0) | 0.5 (1.0) |
| | 0.0625 | 1 (1.0) | 0.5 (1.0) | 0.5 (1.0) |
| | 0.125 | 1 (1.0) | 0.5 (1.0) | 0.5 (1.0) |
| | 2 | | 0.25 (1.0) | |
| | 4 | | 0.016 (1.0) | 0.016 (1.0) |

### Discussion

The results show an additive effect in the combinations of Ramizol and Cefepime against S. aureus ATCC 29213 with a mean of (Σ) FICI of 1.0, Table 10.2.

The results show an additive effect in the combinations of Ramizol and Daptomycin against S. aureus ATCC 29213 with a mean of (Σ) FICI of 1.0, Table 14.2.

The results show an additive effect in the combinations of Chlopam and Oxacillin against S. aureus ATCC 29213 with a mean of (Σ) FICI of 1.0, Table 17.2.

The results show an additive effect in the combinations of Chlopam and Vancomycin against S. aureus ATCC 29213 with a mean of (Σ) FICI of 1.0, Table 18.2.

The results show an additive effect in the combinations of Chlopam and Linezolid against S. aureus ATCC 29213 with a mean of (Σ) FICI of 0.9, Table 19.2.

The results show an additive effect in the combinations of Chlopam and Cefepime against S. aureus BAA-1717 with a mean of (Σ) FICI of 0.8, Table 28.2.

The results show an additive effect in the combinations of Ramizol and Cefepime against S. aureus BAA-1717 with a mean of (Σ) FICI of 0.696, Table 22.2.

The results show an additive effect in the combinations of Ramizol and Oxacillin against S. aureus BAA-1717 with a mean of (Σ) FICI of 0.9, Table 23.2.

The individual antibiotic combinations of Chlopam and Vancomycin, namely 0.003901 μg/mL and 1 μg/mL, 0.0078125 and 1 μg/mL, 0.015625 μg/mL and 1 μg/mL, 0.125 μg/mL and 0.5 μg/mL, and 0.25 μg/mL and 0.5 μg/mL (Table 18-2, entries 2-4, 7-8) led to an additive effect against S. aureus ATCC 29213.

The individual antibiotic combinations of Chlopam and Vancomycin, namely 0.001953 μg/mL and 1 μg/mL, 0.003901 μg/mL and 1 μg/mL, 0.0078125 and 1 μg/mL, and 0.015625 μg/mL and 1 μg/mL (Table 30-2, entries 2-5) led to an additive effect against S. aureus BAA-1717.

The individual antibiotic combinations of Chlopam and Linezolid, namely 0.003901 μg/mL and 2 μg/mL, 0.0078125 and 2 μg/mL, 0.015625 μg/mL and 2 μg/mL, 0.125 μg/mL and 1 μg/mL, 0.25 μg/mL and 0.5 μg/mL, and 0.5 μg/mL and 0.25 μg/mL (Table 19-2, entries 2-4, 7-9) led to an additive effect against S. aureus ATCC 29213.

The individual antibiotic combinations of Chlopam and Linezolid, namely 0.001953 μg/mL and 1 μg/mL, 0.003901 μg/mL and 1 μg/mL, 0.0078125 and 1 μg/mL, and 0.015625 μg/mL and 1 μg/mL (Table 31-2, entries 2-5) led to an additive effect against S. aureus BAA-1717.

The individual antibiotic combinations of Chlopam and daptomycin, namely 0.003901 μg/mL and 0.5 μg/mL, 0.0078125 and 0.5 μg/mL, 0.015625 μg/mL and 0.5 μg/mL, 0.25 μg/mL and 0.25 μg/mL, and 0.5 μg/mL and 0.016 μg/mL (Table 20-2, entries 2-4, 8,12) led to an additive effect against S. aureus ATCC 29213.

The individual antibiotic combinations of Chlopam and daptomycin, namely 0.001953 μg/mL and 0.5 μg/mL, 0.003901 μg/mL and 0.5 μg/mL, 0.0078125 and 0.5 μg/mL, and 0.015625 μg/mL and 0.5 μg/mL, and 0.5 μg/mL and 0.016 μg/mL (Table 32-2, entries 2-5, 14) led to an additive effect against S. aureus BAA-1717.

The individual antibiotic combinations of Chlopam and mupirocin, namely 0.003901 μg/mL and 0.5 μg/mL, 0.0078125 and 0.5 μg/mL, 0.015625 μg/mL and 0.5 μg/mL, and 0.25 μg/mL and 0.25 μg/mL (Table 21-2, entries 2-4, 8) led to an additive effect against S. aureus ATCC 29213.

The individual antibiotic combinations of Chlopam and mupirocin, namely 0.001953 μg/mL and 0.5 μg/mL, 0.003901 μg/mL and 0.5 μg/mL, 0.0078125 and 0.5 μg/mL, and 0.015625 μg/mL and 0.5 μg/mL, and 0.5 μg/mL and 0.016 μg/mL (Table 33-2, entries 2-5, 14) led to an additive effect against S. aureus BAA-1717.

The individual antibiotic combinations of Ramizol and Vancomycin, namely 0.015625 μg/mL and 1 μg/mL, 0.03125 μg/mL and 1 μg/mL, 0.0625 and 1 μg/mL, and 0.125 μg/mL and 1 μg/mL (Table 12-2, entries 2-5) led to an additive effect against S. aureus ATCC 29213.

The individual antibiotic combinations of Ramizol and Vancomycin, namely 0.015625 μg/mL and 1 μg/mL, 0.03125 μg/mL and 1 μg/mL, 0.0625 and 1 μg/mL, 0.125 μg/mL and 1 μg/mL, and 4 μg/mL and 0.031 μg/mL (Table 24-2, entries 2-5, 14) led to an additive effect against S. aureus BAA-1717.

The individual antibiotic combinations of Ramizol and linezolid, namely 0.015625 μg/mL and 4 μg/mL, 0.03125 μg/mL and 4 μg/mL, 0.0625 and 4 μg/mL, 0.125 μg/mL and 4 μg/mL, and 0.25 μg/mL and 4 μg/mL (Table 13-2, entries 2-6) led to an additive effect against S. aureus ATCC 29213.

The individual antibiotic combinations of Ramizol and linezolid, namely 0.015625 μg/mL and 1 μg/mL, 0.03125 μg/mL and 1 μg/mL, 0.0625 and 1 μg/mL, and 0.125 μg/mL and 1 μg/mL (Table 25-2, entries 2-5) led to an additive effect against S. aureus BAA-1717.

The individual antibiotic combinations of Ramizol and daptomycin, namely 0.015625 μg/mL and 0.5 μg/mL, 0.03125 μg/mL and 0.5 μg/mL, 0.0625 and 0.5 μg/mL, 0.125 μg/mL and 0.5 μg/mL, 2 μg/mL and 0.25 μg/mL, 2 μg/mL and 0.125 μg/mL, 2 μg/mL and 0.063 μg/mL, 2 μg/mL and 0.031 μg/mL, and 4 μg/mL and 0.016 μg/mL (Table 14-2, entries 2-5,9-13) led to an additive effect against S. aureus ATCC 29213.

The individual antibiotic combinations of Ramizol and daptomycin, namely 0.015625 μg/mL and 0.5 μg/mL, 0.03125 μg/mL and 0.5 μg/mL, 0.0625 and 0.5 μg/mL, 0.125 μg/mL and 0.5 μg/mL, 2 μg/mL and 0.25 μg/mL, and 4 μg/mL and 0.016 μg/mL (Table 26-2, entries 2-5,9,13) led to an additive effect against S. aureus BAA-1717.

The individual antibiotic combinations of Ramizol and mupirocin, namely 0.015625 μg/mL and 0.5 μg/mL, 0.03125 μg/mL and 0.5 μg/mL, 0.0625 and 0.5 μg/mL, 0.125 μg/mL and 0.5 μg/mL, and 0.25 μg/mL and 0.5 μg/mL (Table 15-2, entries 2-6) led to an additive effect against S. aureus ATCC 29213.

The individual antibiotic combinations of Ramizol and mupirocin, namely 0.015625 μg/mL and 0.5 μg/mL, 0.03125 μg/mL and 0.5 μg/mL, 0.0625 and 0.5 μg/mL, 0.125 μg/mL and 0.5 μg/mL, and 4 μg/mL and 0.016 μg/mL (Table 27-2, entries 2-5,14) led to an additive effect against S. aureus BAA-1717.

The individual antibiotic combinations of lower concentrations of Chlopam and Oxacillin, namely 0.0125 μg/mL and 0.125 μg/mL (Table 11-2, entry 8) led to a synergistic effect against S. aureus ATCC 29213.

The individual antibiotic combinations of lower concentrations of Chlopam and Cefepime, namely 0.25 μg/mL and 64 μg/mL, 0.25 μg/mL and 32 μg/mL, 0.25 μg/mL and 16 μg/mL, and 0.50 μg/mL and 8 μg/mL (Table 28-2, entries 9-12) led to a synergistic effect against S. aureus ATCC BAA-1717.

The individual antibiotic combinations of lower concentrations of Ramizol and Cefepime, namely 0.125 μg/mL and 128 μg/mL, 1 μg/mL and 64 μg/mL, and 2 μg/mL and 8 μg/mL (Table 22-2, entries 5,8,11) led to a synergistic effect against S. aureus ATCC BAA-1717.

### REFERENCES

CLSI. Methods for Dilution In Antimicrobial Susceptibility Tests for Bacteria That Grow Aerobically; Approved Standard-Eighth Edition. CLSI document M07-A8 (ISBN 1-56238-689-1). Vol. 29 No. 2. Clinical and Laboratory Standards Institute, USA, 2009.

1. A composition comprising a combination of:
(i) at least one aryl antibiotic of Group I wherein Group I consists of: 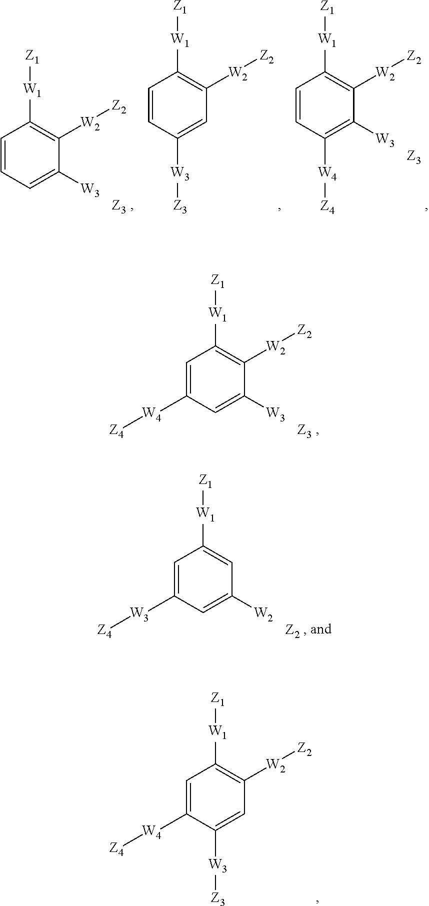
wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of: 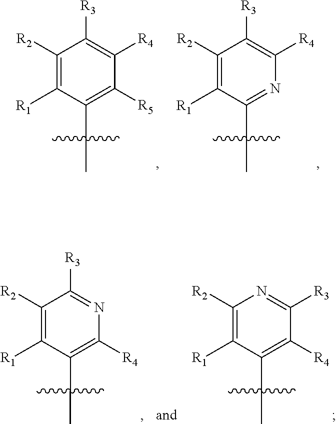
and
each of R₁, R₂, R₃, R₄, and R₅ is independently C₁₋₈ heteroalkyl, and the C₁₋₈ heteroalkyl comprises CO₂H or an ester thereof, with the proviso that at least one of R₁, R₂, R₃, R₄, and R₅ is C₁₋₈ heteroalkyl,
or pharmaceutically acceptable salts thereof; and
(ii) at least one antibiotic that comprises penicillin or a derivative thereof, cepham or a derivative thereof, vancomycin, linezolid, daptomycin or mupirocin.

2. A method for suppressing, inhibiting, preventing, alleviating or treating a bacterial infection, the method comprising the step of:
a) administering to a subject a composition according to claim 1.

3. A method to control or manipulate a commensal bacterial population, the method comprising the step of:
a) administering to a subject a composition according to claim 1.

4. A method to delay or prevent the development of antibiotic resistance in a bacteria to the aryl antibiotics of Group I or pharmaceutically acceptable salts thereof and/or the antibiotics selected from the group consisting of penicillin or a derivative thereof, cepham or a derivative thereof, vancomycin, linezolid, daptomycin or mupirocin, the method comprising the step of:
a) administering to a subject a composition according to claim 1.

5. A method to prevent, remove or control bacterial contamination, the method comprising the step of:
a) applying a composition according to claim 1 to a surface of a subject.

6. A method of making a composition according to claim 1, the method comprising mixing at least one aryl antibiotic of Group I or at least one pharmaceutically acceptable salt thereof with at least one antibiotic comprising penicillin or a derivative thereof, cepham or a derivative thereof, vancomycin, linezolid, daptomycin or mupirocin.

7. The method of claim 2, wherein the bacteria is a Staphylococcus species.

8. The composition according to claim 1, further comprising a pharmaceutically acceptable carrier, diluent, filler, polymer, glidant, lubricant, or excipient.

9. The composition of claim 1, wherein the aryl antibiotic of Group I is an aryl antibiotic of Formula A 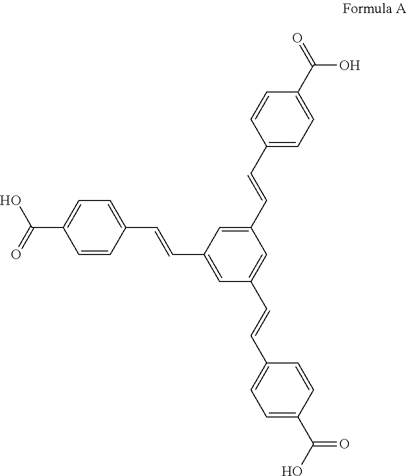 [SMILES: O=C(O)c1ccc(/C=C/c2cc(/C=C/c3ccc(C(=O)O)cc3)cc(/C=C/c3ccc(C(=O)O)cc3)c2)cc1]
or Formula B 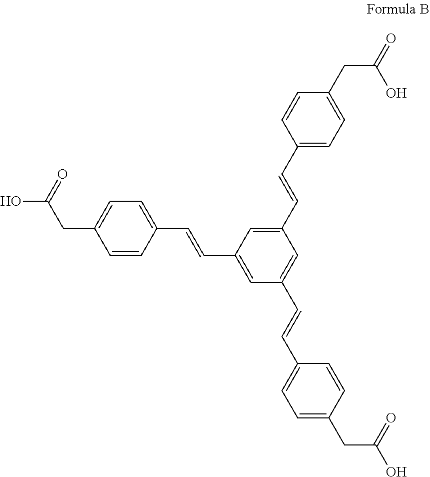 [SMILES: O=C(O)Cc1ccc(/C=C/c2cc(/C=C/c3ccc(CC(=O)O)cc3)cc(/C=C/c3ccc(CC(=O)O)cc3)c2)cc1]
or pharmaceutically acceptable salts thereof.

10. The composition of claim 1, wherein the at least one antibiotic of (ii) comprises penicillin or a derivative thereof or cepham or a derivative thereof.

11. The composition of claim 1, wherein the at least one antibiotic of (ii) comprises. oxacillin.

12. The composition of claim 1, wherein the at least one antibiotic of (ii) comprises cefepime.

13. The method of claim 2, wherein the aryl antibiotic of Group I is an aryl antibiotic of Formula A 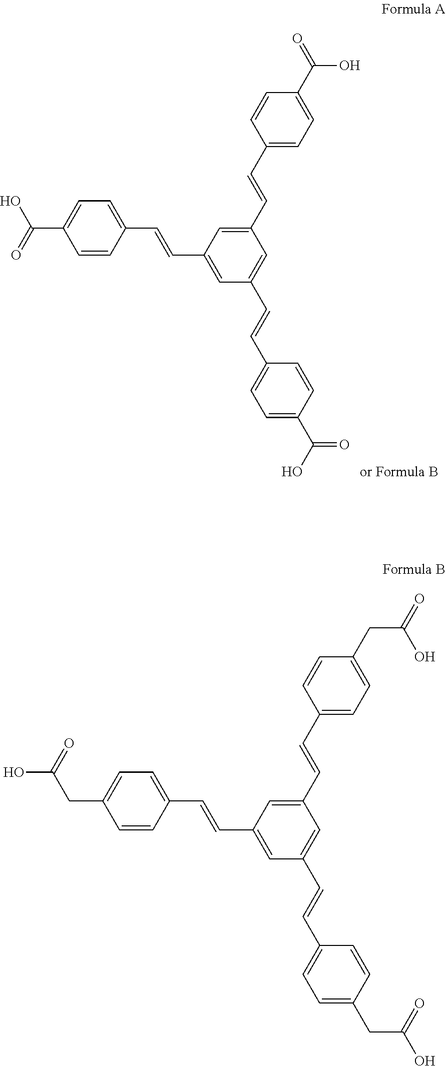 [SMILES: O=C(O)c1ccc(/C=C/c2cc(/C=C/c3ccc(C(=O)O)cc3)cc(/C=C/c3ccc(C(=O)O)cc3)c2)cc1 ; O=C(O)Cc1ccc(/C=C/c2cc(/C=C/c3ccc(CC(=O)O)cc3)cc(/C=C/c3ccc(CC(=O)O)cc3)c2)cc1]
or a pharmaceutically acceptable salt thereof.

14. The method of claim 2, wherein the at least one antibiotic of (ii) comprises penicillin or a derivative thereof or cepham or a derivative thereof.

15. The method of claim 2, wherein the at least one antibiotic of (ii) comprises oxacillin.

16. The method of claim 2, wherein the at least one antibiotic of (ii) comprises cefepime.
